SEQUENCE LISTING (136 sequences)

SEQ ID NO: 1 - DNA, 50 bp, synthetic construct
  misc_feature 1..50: note = Sequences Detected for each Gene Target for Staphylococcus aureus in Dilution Series speH 6070 seq_17
  source 1..50: mol_type = other DNA
aatatttagttaactcattaacgttagtttcgttttgatttatataataa

SEQ ID NO: 2 - DNA, 50 bp, synthetic construct
  misc_feature 1..50: note = Sequences Detected for each Gene Target for Staphylococcus aureus in Dilution Series rot 67299 seq_16
  source 1..50: mol_type = other DNA
ttagaaacacttttgggtgacattaactcaattttcagcgagattgaaag

SEQ ID NO: 3 - DNA, 50 bp, synthetic construct
  misc_feature 1..50: note = Sequences Detected for each Gene Target for Staphylococcus aureus in Dilution Series femC 521517 seq_15
  source 1..50: mol_type = other DNA
ttcatctgttgttaaatgctcttgtgagtcataaatgatttgtttaatcc

SEQ ID NO: 4 - DNA, 50 bp, synthetic construct; identical to SEQ ID NO: 1
  misc_feature 1..50: note = Sequences Detected for each Gene Target for Staphylococcus aureus in Dilution Series speH 9087 seq_17
  source 1..50: mol_type = other DNA

SEQ ID NO: 5 - DNA, 50 bp, synthetic construct; identical to SEQ ID NO: 2
  misc_feature 1..50: note = Sequences Detected for each Gene Target for Staphylococcus aureus in Dilution Series rot 74752 seq_16
  source 1..50: mol_type = other DNA

SEQ ID NO: 6 - DNA, 43 bp, synthetic construct
  misc_feature 1..43: note = Sequences Detected for each Gene Target for Staphylococcus aureus in Dilution Series lpl3_1 99 seq_34
  source 1..43: mol_type = other DNA
atcacttttgccacaacctgctatcgcaaatattaaaatcata

SEQ ID NO: 7 - DNA, 50 bp, synthetic construct; identical to SEQ ID NO: 3
  misc_feature 1..50: note = Sequences Detected for each Gene Target for Staphylococcus aureus in Dilution Series femC 457053 seq_15
  source 1..50: mol_type = other DNA

SEQ ID NO: 8 - DNA, 50 bp, synthetic construct; identical to SEQ ID NO: 1
  misc_feature 1..50: note = Sequences Detected for each Gene Target for Staphylococcus aureus in Dilution Series speH 3441 seq_17
  source 1..50: mol_type = other DNA

SEQ ID NO: 9 - DNA, 50 bp, synthetic construct; identical to SEQ ID NO: 2
  misc_feature 1..50: note = Sequences Detected for each Gene Target for Staphylococcus aureus in Dilution Series rot 66226 seq_16
  source 1..50: mol_type = other DNA

SEQ ID NO: 10 - DNA, 43 bp, synthetic construct; identical to SEQ ID NO: 6
  misc_feature 1..43: note = Sequences Detected for each Gene Target for Staphylococcus aureus in Dilution Series lpl3_1 240 seq_34
  source 1..43: mol_type = other DNA

SEQ ID NO: 11 - DNA, 50 bp, synthetic construct; identical to SEQ ID NO: 3
  misc_feature 1..50: note = Sequences Detected for each Gene Target for Staphylococcus aureus in Dilution Series femC 482885 seq_15
  source 1..50: mol_type = other DNA

SEQ ID NO: 12 - DNA, 50 bp, synthetic construct; identical to SEQ ID NO: 1
  misc_feature 1..50: note = Sequences Detected for each Gene Target for Staphylococcus aureus in Dilution Series speH 16980 seq_17
  source 1..50: mol_type = other DNA

SEQ ID NO: 13 - DNA, 50 bp, synthetic construct; identical to SEQ ID NO: 2
  misc_feature 1..50: note = Sequences Detected for each Gene Target for Staphylococcus aureus in Dilution Series rot 56168 seq_16
  source 1..50: mol_type = other DNA

SEQ ID NO: 14 - DNA, 43 bp, synthetic construct; identical to SEQ ID NO: 6
  misc_feature 1..43: note = Sequences Detected for each Gene Target for Staphylococcus aureus in Dilution Series lpl3_1 9888 seq_34
  source 1..43: mol_type = other DNA

SEQ ID NO: 15 - DNA, 50 bp, synthetic construct; identical to SEQ ID NO: 3
  misc_feature 1..50: note = Sequences Detected for each Gene Target for Staphylococcus aureus in Dilution Series femC 111120 seq_15
  source 1..50: mol_type = other DNA

SEQ ID NO: 16 - DNA, 50 bp, synthetic construct; identical to SEQ ID NO: 1
  misc_feature 1..50: note = Sequences Detected for each Gene Target for Staphylococcus aureus in Dilution Series speH 12653 seq_17
  source 1..50: mol_type = other DNA

SEQ ID NO: 17 - DNA, 50 bp, synthetic construct; identical to SEQ ID NO: 2
  misc_feature 1..50: note = Sequences Detected for each Gene Target for Staphylococcus aureus in Dilution Series rot 17078 seq_16
  source 1..50: mol_type = other DNA

SEQ ID NO: 18 - DNA, 43 bp, synthetic construct; identical to SEQ ID NO: 6
  misc_feature 1..43: note = Sequences Detected for each Gene Target for Staphylococcus aureus in Dilution Series lpl3_1 13588 seq_34
  source 1..43: mol_type = other DNA

SEQ ID NO: 19 - DNA, 50 bp, synthetic construct; identical to SEQ ID NO: 3
  misc_feature 1..50: note = Sequences Detected for each Gene Target for Staphylococcus aureus in Dilution Series femC 26677 seq_15
  source 1..50: mol_type = other DNA

SEQ ID NO: 20 - DNA, 50 bp, synthetic construct; identical to SEQ ID NO: 1
  misc_feature 1..50: note = Sequences Detected for each Gene Target for Staphylococcus aureus in Dilution Series speH 8674 seq_17
  source 1..50: mol_type = other DNA

SEQ ID NO: 21 - DNA, 50 bp, synthetic construct; identical to SEQ ID NO: 2
  misc_feature 1..50: note = Sequences Detected for each Gene Target for Staphylococcus aureus in Dilution Series rot 6212 seq_16
  source 1..50: mol_type = other DNA

SEQ ID NO: 22 - DNA, 43 bp, synthetic construct; identical to SEQ ID NO: 6
  misc_feature 1..43: note = Sequences Detected for each Gene Target for Staphylococcus aureus in Dilution Series lpl3_1 15824 seq_34
  source 1..43: mol_type = other DNA

SEQ ID NO: 23 - DNA, 50 bp, synthetic construct; identical to SEQ ID NO: 3
  misc_feature 1..50: note = Sequences Detected for each Gene Target for Staphylococcus aureus in Dilution Series femC 3106 seq_15
  source 1..50: mol_type = other DNA

SEQ ID NO: 24 - DNA, 50 bp, synthetic construct; identical to SEQ ID NO: 1
  misc_feature 1..50: note = Sequences Detected for each Gene Target for Staphylococcus aureus in Dilution Series speH 37662 seq_17
  source 1..50: mol_type = other DNA

SEQ ID NO: 25 - DNA, 50 bp, synthetic construct; identical to SEQ ID NO: 2
  misc_feature 1..50: note = Sequences Detected for each Gene Target for Staphylococcus aureus in Dilution Series rot 28571 seq_16
  source 1..50: mol_type = other DNA

SEQ ID NO: 26 - DNA, 43 bp, synthetic construct; identical to SEQ ID NO: 6
  misc_feature 1..43: note = Sequences Detected for each Gene Target for Staphylococcus aureus in Dilution Series 31169 seq_34
  source 1..43: mol_type = other DNA

SEQ ID NO: 27 - DNA, 50 bp, synthetic construct
  misc_feature 1..50: note = Sequences Detected for each Gene Target for Pseudomonas aeruginosa pslM 140 seq_12
  source 1..50: mol_type = other DNA
tctggcacctgctgcgctacggtcgcgccatgcacctggtcaatggcgtg

SEQ ID NO: 28 - DNA, 50 bp, synthetic construct
  misc_feature 1..50: note = Sequences Detected for each Gene Target for Pseudomonas aeruginosa mucA 29850 seq_11
  source 1..50: mol_type = other DNA
aagggaccgtggcggatggtcggtcgcctggcggtcgctgcctcggtgac

SEQ ID NO: 29 - DNA, 50 bp, synthetic construct
  misc_feature 1..50: note = Sequences Detected for each Gene Target for Pseudomonas aeruginosa iscR 392 seq_31
  source 1..50: mol_type = other DNA
ccggaccttgctgagcatgcagcgccaggtcgagcatggcggttacggcg

SEQ ID NO: 30 - DNA, 50 bp, synthetic construct
  misc_feature 1..50: note = Sequences Detected for each Gene Target for Pseudomonas aeruginosa fecI_5 386 seq_10
  source 1..50: mol_type = other DNA
agcacctgggcagttcggcggacgccgccgacctcgcccaggatactttc

SEQ ID NO: 31 - DNA, 50 bp, synthetic construct
  misc_feature 1..50: note = Sequences Detected for each Gene Target for Pseudomonas aeruginosa fecI_12 107 seq_33
  source 1..50: mol_type = other DNA
ccgaagacctcgccgccgaggccttcctgcaggtctggatgctccccgac

SEQ ID NO: 32 - DNA, 50 bp, synthetic construct
  misc_feature 1..50: note = Sequences Detected for each Gene Target for Pseudomonas aeruginosa fecI_11 155 seq_32
  source 1..50: mol_type = other DNA
gccgaggacatcgcctcggaaaccttcctcaaggtccttgccctgccgga

SEQ ID NO: 33 - DNA, 50 bp, synthetic construct
  misc_feature 1..50: note = Sequences Detected for each Gene Target for Pseudomonas aeruginosa dksA_1 255 seq_9
  source 1..50: mol_type = other DNA
ctacggctggtgccaggaaaccggcgagccgatcggcctgcgccgcctgc

SEQ ID NO: 34 - DNA, 50 bp, synthetic construct; identical to SEQ ID NO: 27
  misc_feature 1..50: note = Sequences Detected for each Gene Target for Pseudomonas aeruginosa pslM 92 seq_12
  source 1..50: mol_type = other DNA

SEQ ID NO: 35 - DNA, 50 bp, synthetic construct; identical to SEQ ID NO: 28
  misc_feature 1..50: note = Sequences Detected for each Gene Target for Pseudomonas aeruginosa mucA 29904 seq_11
  source 1..50: mol_type = other DNA

SEQ ID NO: 36 - DNA, 50 bp, synthetic construct; identical to SEQ ID NO: 29
  misc_feature 1..50: note = Sequences Detected for each Gene Target for Pseudomonas aeruginosa iscR 297 seq_31
  source 1..50: mol_type = other DNA

SEQ ID NO: 37 - DNA, 50 bp, synthetic construct; identical to SEQ ID NO: 30
  misc_feature 1..50: note = Sequences Detected for each Gene Target for Pseudomonas aeruginosa fecI_5 372 seq_10
  source 1..50: mol_type = other DNA

SEQ ID NO: 38 - DNA, 50 bp, synthetic construct; identical to SEQ ID NO: 31
  misc_feature 1..50: note = Sequences Detected for each Gene Target for Pseudomonas aeruginosa fecI_12 168 seq_33
  source 1..50: mol_type = other DNA

SEQ ID NO: 39 - DNA, 50 bp, synthetic construct; identical to SEQ ID NO: 32
  misc_feature 1..50: note = Sequences Detected for each Gene Target for Pseudomonas aeruginosa fecI_11 225 seq_32
  source 1..50: mol_type = other DNA

SEQ ID NO: 40 - DNA, 50 bp, synthetic construct; identical to SEQ ID NO: 33
  misc_feature 1..50: note = Sequences Detected for each Gene Target for Pseudomonas aeruginosa dksA_1 357 seq_9
  source 1..50: mol_type = other DNA

SEQ ID NO: 41 - DNA, 50 bp, synthetic construct; identical to SEQ ID NO: 27
  misc_feature 1..50: note = Sequences Detected for each Gene Target for Pseudomonas aeruginosa pslM 299 seq_12
  source 1..50: mol_type = other DNA

SEQ ID NO: 42 - DNA, 50 bp, synthetic construct; identical to SEQ ID NO: 28
  misc_feature 1..50: note = Sequences Detected for each Gene Target for Pseudomonas aeruginosa mucA 21357 seq_11
  source 1..50: mol_type = other DNA

SEQ ID NO: 43 - DNA, 50 bp, synthetic construct; identical to SEQ ID NO: 29
  misc_feature 1..50: note = Sequences Detected for each Gene Target for Pseudomonas aeruginosa iscR 240 seq_31
  source 1..50: mol_type = other DNA

SEQ ID NO: 44 - DNA, 50 bp, synthetic construct; identical to SEQ ID NO: 27
  misc_feature 1..50: note = Sequences Detected for each Gene Target for Pseudomonas aeruginosa pslM 1297 seq_12
  source 1..50: mol_type = other DNA

SEQ ID NO: 45 - DNA, 50 bp, synthetic construct; identical to SEQ ID NO: 28
  misc_feature 1..50: note = Sequences Detected for each Gene Target for Pseudomonas aeruginosa mucA 18358 seq_11
  source 1..50: mol_type = other DNA

SEQ ID NO: 46 - DNA, 50 bp, synthetic construct
  misc_feature 1..50: note = Sequences Detected for each Gene Target for Pseudomonas aeruginosa fecI_12 1054 seq_30
  source 1..50: mol_type = other DNA
ccgaagacctcgccgccgaggccttcctacaggtctggatgctccccgac

SEQ ID NO: 47 - DNA, 50 bp, synthetic construct; identical to SEQ ID NO: 28
  misc_feature 1..50: note = Sequences Detected for each Gene Target for Pseudomonas aeruginosa mucA 8477 seq_11
  source 1..50: mol_type = other DNA

SEQ ID NO: 48 - DNA, 50 bp, synthetic construct; identical to SEQ ID NO: 46
  misc_feature 1..50: note = Sequences Detected for each Gene Target for Pseudomonas aeruginosa fecI_12 1932 seq_30
  source 1..50: mol_type = other DNA

SEQ ID NO: 49 - DNA, 50 bp, synthetic construct
  misc_feature 1..50: note = Sequences Detected for each Gene Target for Pseudomonas aeruginosa dksA_1 1434 seq_29
  source 1..50: mol_type = other DNA
agaagctgctcgacaagatcgacgaagcgctggagcgcctggcccgcggc

SEQ ID NO: 50 - DNA, 50 bp, synthetic construct
  misc_feature 1..50: note = Sequences Detected for each Gene Target for Pseudomonas aeruginosa ptlF_4 1348 seq_28
  source 1..50: mol_type = other DNA
cgccggctggtggtgatgactaggttggtcttgaggtccgatcggatcgg

SEQ ID NO: 51 - DNA, 50 bp, synthetic construct; identical to SEQ ID NO: 30
  misc_feature 1..50: note = Sequences Detected for each Gene Target for Pseudomonas aeruginosa fecI_5 2110 seq_10
  source 1..50: mol_type = other DNA

SEQ ID NO: 52 - DNA, 50 bp, synthetic construct
  misc_feature 1..50: note = Sequences Detected for each Gene Target for Pseudomonas aeruginosa fecI_11 1993 seq_13
  source 1..50: mol_type = other DNA
gccgaggacatcgcctcggaaaccttcctcaaggtcctcgccctgccgga

SEQ ID NO: 53 - DNA, 50 bp, synthetic construct
  misc_feature 1..50: note = Candida albicans Sequences Detected for each Gene Target in the Fungal Pool MEY_03361 19094 seq_21
  source 1..50: mol_type = other DNA
gcattagcatataaaactttacatcgtaatcaaaaacgaccaccattgcc

SEQ ID NO: 54 - DNA, 50 bp, synthetic construct
  misc_feature 1..50: note = Candida albicans Sequences Detected for each Gene Target in the Fungal Pool MEY_04223 24288 seq_22
  source 1..50: mol_type = other DNA
agatgctaagcctggtaataaaaaggactgattagtgtcagtgatatgtg

SEQ ID NO: 55 - DNA, 50 bp, synthetic construct
  misc_feature 1..50: note = Candida albicans Sequences Detected for each Gene Target in the Fungal Pool MEY_05088 15845 seq_24
  source 1..50: mol_type = other DNA
gagttggacgatgacaacattttcttatggaatgttggtgtaatggtctt

SEQ ID NO: 56 - DNA, 50 bp, synthetic construct
  misc_feature 1..50: note = Candida albicans Sequences Detected for each Gene Target in the Fungal Pool MEY_06242 17000 seq_35
  source 1..50: mol_type = other DNA
tatgttaatcctaagactatggcatttggacaaatttatcatggtgttca

SEQ ID NO: 57 - DNA, 50 bp, synthetic construct
  misc_feature 1..50: note = Aspergillus fumigatus Gene Target Sequences Detected in the Fungal Pool for each Gene AFUA_1G05430 2215 seq_33
  source 1..50: mol_type = other DNA
ctgcaatccaagtcctccacaaccttcaacatcagcatctttggacctct

SEQ ID NO: 58 - DNA, 50 bp, synthetic construct
  misc_feature 1..50: note = Aspergillus fumigatus Gene Target Sequences Detected in the Fungal Pool for each Gene AFUA_8G00910 2956 seq_34
  source 1..50: mol_type = other DNA
ggcatagcatgatggtagtcgacctcatacagcgtcgccgcgttcgggtt

SEQ ID NO: 59 - DNA, 50 bp, synthetic construct
  misc_feature 1..50: note = Aspergillus fumigatus Gene Target Sequences Detected in the Fungal Pool for each Gene AFUA_8G02500 5840 seq_35
  source 1..50: mol_type = other DNA
ccctggatactggctccgaagggatggctgtcttgacaacgccgcgatga

SEQ ID NO: 60 - DNA, 50 bp, synthetic construct
  misc_feature 1..50: note = Candida glabrata Gene Target Sequences Detected in the Fungal Pool for each Gene CAGL0A00187g 9986 seq_36
  source 1..50: mol_type = other DNA
ggcgaggtaaacaacaaggatttaccagcaatggtattaataaagatcaa

SEQ ID NO: 61 - DNA, 50 bp, synthetic construct
  misc_feature 1..50: note = Candida glabrata Gene Target Sequences Detected in the Fungal Pool for each Gene CAGL0A00297g 14417 seq_37
  source 1..50: mol_type = other DNA
atactccttgttatcgtagcattcctttaattctgcttctattttttcta

SEQ ID NO: 62 - DNA, 50 bp, synthetic construct
  misc_feature 1..50: note = Candida glabrata Gene Target Sequences Detected in the Fungal Pool for each Gene CAGL0A00803g 14787 seq_38
  source 1..50: mol_type = other DNA
tggatgatgatgagccgatgcagcgctatatcctacagtctctgttttcg

SEQ ID NO: 63 - DNA, 50 bp, synthetic construct; identical to SEQ ID NO: 33
  misc_feature 1..50: note = Pseudomonas aeruginosa Gene Target Sequences Detected in Patients from Table 10. fecI_5 312 seq_10
  source 1..50: mol_type = other DNA

SEQ ID NO: 64 - DNA, 50 bp, synthetic construct; identical to SEQ ID NO: 30
  misc_feature 1..50: note = Pseudomonas aeruginosa Gene Target Sequences Detected in Patients from Table 10. mucA 3654 seq_11
  source 1..50: mol_type = other DNA

SEQ ID NO: 65 - DNA, 50 bp, synthetic construct; identical to SEQ ID NO: 28
  misc_feature 1..50: note = Pseudomonas aeruginosa Gene Target Sequences Detected in Patients from Table 10. dksA_1 2772 seq_9
  source 1..50: mol_type = other DNA

SEQ ID NO: 66 - DNA, 50 bp, synthetic construct; identical to SEQ ID NO: 33
  misc_feature 1..50: note = Pseudomonas aeruginosa Gene Target Sequences Detected in Patients from Table 10. fecI_5 956 seq_10
  source 1..50: mol_type = other DNA

SEQ ID NO: 67 - DNA, 50 bp, synthetic construct; identical to SEQ ID NO: 30
  misc_feature 1..50: note = Pseudomonas aeruginosa Gene Target Sequences Detected in Patients from Table 10. mucA 11889 seq_11
  source 1..50: mol_type = other DNA

SEQ ID NO: 68 - DNA, 50 bp, synthetic construct; identical to SEQ ID NO: 28
  misc_feature 1..50: note = Pseudomonas aeruginosa Gene Target Sequences Detected in Patients from Table 10. pslM 67 seq_12
  source 1..50: mol_type = other DNA

SEQ ID NO: 69 - DNA, 50 bp, synthetic construct; identical to SEQ ID NO: 27
  misc_feature 1..50: note = Pseudomonas aeruginosa Gene Target Sequences Detected in Patients from Table 10. dksA_1 19669 seq_9
  source 1..50: mol_type = other DNA

SEQ ID NO: 70 - DNA, 50 bp, synthetic construct; identical to SEQ ID NO: 33
  misc_feature 1..50: note = Pseudomonas aeruginosa Gene Target Sequences Detected in Patients from Table 10. fecI_11 189 seq_13
  source 1..50: mol_type = other DNA

SEQ ID NO: 71 - DNA, 50 bp, synthetic construct; identical to SEQ ID NO: 52
  misc_feature 1..50: note = Pseudomonas aeruginosa Gene Target Sequences Detected in Patients from Table 10. fecI_5 7826 seq_10
  source 1..50: mol_type = other DNA

SEQ ID NO: 72 - DNA, 50 bp, synthetic construct; identical to SEQ ID NO: 30
  misc_feature 1..50: note = Pseudomonas aeruginosa Gene Target Sequences Detected in Patients from Table 10. iscR 1098 seq_14
  source 1..50: mol_type = other DNA

SEQ ID NO: 73 - DNA, 50 bp, synthetic construct
  misc_feature 1..50: note = Pseudomonas aeruginosa Gene Target Sequences Detected in Patients from Table 10. mucA 18647 seq_11
  source 1..50: mol_type = other DNA
ccgccatgctcgacctggcgctgcatgctcagcaaggtccggtctcgctg

SEQ ID NO: 74 - DNA, 50 bp, synthetic construct; identical to SEQ ID NO: 28
  misc_feature 1..50: note = Pseudomonas aeruginosa Gene Target Sequences Detected in Patients from Table 10. pslM 669 seq_12
  source 1..50: mol_type = other DNA

SEQ ID NO: 75 - DNA, 50 bp, synthetic construct; identical to SEQ ID NO: 27
  misc_feature 1..50: note = Pseudomonas aeruginosa Gene Target Sequences Detected in Patients from Table 10.
  source 1..50: mol_type = other DNA

SEQ ID NO: 76 - DNA, 50 bp, synthetic construct
  misc_feature 1..50: note = Haemophilus parainfluenzae Gene Target Sequences Detected in Patients from Table 10. 16s 21232 seq_55
  source 1..50: mol_type = other DNA
agcgagagtgcgagctggagcgaatctcacaaagtacgtctaagtccgga

SEQ ID NO: 77 - DNA, 50 bp, synthetic construct
  misc_feature 1..50: note = Haemophilus parainfluenzae Gene Target Sequences Detected in Patients from Table 10. 16s 15949 seq_56
  source 1..50: mol_type = other DNA
aagcgatagtgcgagctggagcgaatctcacaaagtacgtctaagtccgg

SEQ ID NO: 78 - DNA, 50 bp, synthetic construct
  misc_feature 1..50: note = Haemophilus parainfluenzae Gene Target Sequences Detected in Patients from Table 10.
  source 1..50: mol_type = other DNA
aagcgagagtgcgagctggagcgaatctcacaaagtacgtctaagtccgg

SEQ ID NO: 79 - DNA, 50 bp, synthetic construct
  misc_feature 1..50: note = Haemophilus influenzae Gene Target Sequences Detected in Patients from Table 10. hgpC 410 seq_1
  source 1..50: mol_type = other DNA
gagcttaaggtttcagcttgacgaagcccatcaaccataatacctacacg

SEQ ID NO: 80 - DNA, 50 bp, synthetic construct
  misc_feature 1..50: note = Haemophilus influenzae Gene Target Sequences Detected in Patients from Table 10. relB 242 seq_2
  source 1..50: mol_type = other DNA
caatatactgtttattaaatcgtttacctttacccgaaaactaccaagat

SEQ ID NO: 81 - DNA, 50 bp, synthetic construct
  misc_feature 1..50: note = Haemophilus influenzae Gene Target Sequences Detected in Patients from Table 10. uppB 97 seq_3
  source 1..50: mol_type = other DNA
actgccacagaatgttatttattcgaaggtgaagggcatttgaataagta

SEQ ID NO: 82 - DNA, 50 bp, synthetic construct
  misc_feature 1..50: note = Haemophilus influenzae Gene Target Sequences Detected in Patients from Table 10. hgpC 9071 seq_4
  source 1..50: mol_type = other DNA
gagcttaaggtttcagcttgacgaagcccatcaaccataatacctacgcg

SEQ ID NO: 83 - DNA, 50 bp, synthetic construct; identical to SEQ ID NO: 80
  misc_feature 1..50: note = Haemophilus influenzae Gene Target Sequences Detected in Patients from Table 10. relB 4625 seq_2
  source 1..50: mol_type = other DNA

SEQ ID NO: 84 - DNA, 50 bp, synthetic construct; identical to SEQ ID NO: 81
  misc_feature 1..50: note = Haemophilus influenzae Gene Target Sequences Detected in Patients from Table 10. uppB 3209 seq_3
  source 1..50: mol_type = other DNA

SEQ ID NO: 85 - DNA, 50 bp, synthetic construct
  misc_feature 1..50: note = Haemophilus influenzae Gene Target Sequences Detected in Patients from Table 10. vapD 9268 seq_5
  source 1..50: mol_type = other DNA
tttgttagaacacaaggaagtttatataccaacatgaatgaagatatggc

SEQ ID NO: 86 - DNA, 50 bp, synthetic construct
  misc_feature 1..50: note = Coronavirus HKU1 Gene Target Sequences Detected in Patients from Table 10. Replicase 1B 972458 seq_25
  source 1..50: mol_type = other DNA
aacacctgtcattgaagccaaagcatctaaaacaagatctgcttttactt

SEQ ID NO: 87 - DNA, 50 bp, synthetic construct
  misc_feature 1..50: note = Coronavirus HKU1 Gene Target Sequences Detected in Patients from Table 10. Spike 27542 seq_26
  source 1..50: mol_type = other DNA
tacccaaaccataagaaacatccacaacatactcacttatgcgaggaact

SEQ ID NO: 88 - DNA, 50 bp, synthetic construct; identical to SEQ ID NO: 79
  misc_feature 1..50: note = Haemophilus influenzae Gene Target Sequences Detected for Patients in Table 19 hgpC 1670.441 seq_1
  source 1..50: mol_type = other DNA

SEQ ID NO: 89 - DNA, 50 bp, synthetic construct; identical to SEQ ID NO: 80
  misc_feature 1..50: note = Haemophilus influenzae Gene Target Sequences Detected for Patients in Table 19 relB 2059.991 seq_2
  source 1..50: mol_type = other DNA

SEQ ID NO: 90 - DNA, 45 bp, synthetic construct
  misc_feature 1..45: note = Haemophilus influenzae Gene Target Sequences Detected for Patients in Table 19 vapD 72284.54 seq_6
  source 1..45: mol_type = other DNA
aagattatcatccaaagggcgttcaagaggcttatacagacattc

SEQ ID NO: 91 - DNA, 50 bp, synthetic construct; identical to SEQ ID NO: 82
  misc_feature 1..50: note = Haemophilus influenzae Gene Target Sequences Detected for Patients in Table 19 hgpC 2809.969 seq_4
  source 1..50: mol_type = other DNA

SEQ ID NO: 92 - DNA, 50 bp, synthetic construct; identical to SEQ ID NO: 80
  misc_feature 1..50: note = Haemophilus influenzae Gene Target Sequences Detected for Patients in Table 19 relB 1630.961 seq_2
  source 1..50: mol_type = other DNA

SEQ ID NO: 93 - DNA, 50 bp, synthetic construct; identical to SEQ ID NO: 81
  misc_feature 1..50: note = Haemophilus influenzae Gene Target Sequences Detected for Patients in Table 19 uppB 1139.708 seq_3
  source 1..50: mol_type = other DNA

SEQ ID NO: 94 - DNA, 50 bp, synthetic construct; identical to SEQ ID NO: 85
  misc_feature 1..50: note = Haemophilus influenzae Gene Target Sequences Detected for Patients in Table 19 vapD 2358.016 seq_5
  source 1..50: mol_type = other DNA

SEQ ID NO: 95 - DNA, 50 bp, synthetic construct
  misc_feature 1..50: note = Haemophilus influenzae Gene Target Sequences Detected for Patients in Table 19 hgpC 103.5307 seq_7
  source 1..50: mol_type = other DNA
gagcttaaagtttcagcttgacgaagtccatcaaccataatacctacgcg

SEQ ID NO: 96 - DNA, 50 bp, synthetic construct; identical to SEQ ID NO: 80
  misc_feature 1..50: note = Haemophilus influenzae Gene Target Sequences Detected for Patients in Table 19 relB 107.0674 seq_2
  source 1..50: mol_type = other DNA

SEQ ID NO: 97 - DNA, 50 bp, synthetic construct; identical to SEQ ID NO: 81
  misc_feature 1..50: note = Haemophilus influenzae Gene Target Sequences Detected for Patients in Table 19 uppB 123.4651 seq_3
  source 1..50: mol_type = other DNA

SEQ ID NO: 98 - DNA, 50 bp, synthetic construct; identical to SEQ ID NO: 85
  misc_feature 1..50: note = Haemophilus influenzae Gene Target Sequences Detected for Patients in Table 19 vapD 535.9801 seq_5
  source 1..50: mol_type = other DNA

SEQ ID NO: 99 - DNA, 50 bp, synthetic construct
  misc_feature 1..50: note = Haemophilus influenzae Gene Target Sequences Detected for Patients in Table 19 yafQ_1 202.56 seq_8
  source 1..50: mol_type = other DNA
ttggaaaggttggcgagattgccatattaagaatgatcttgtgcttattt

SEQ ID NO: 100 - DNA, 50 bp, synthetic construct; identical to SEQ ID NO: 82
  misc_feature 1..50: note = Haemophilus influenzae Gene Target Sequences Detected for Patients in Table 19 hgpC 9070.548 seq_4
  source 1..50: mol_type = other DNA

SEQ ID NO: 101 - DNA, 50 bp, synthetic construct; identical to SEQ ID NO: 80
  misc_feature 1..50: note = Haemophilus influenzae Gene Target Sequences Detected for Patients in Table 19 relB 4625.379 seq_2
  source 1..50: mol_type = other DNA

SEQ ID NO: 102 - DNA, 50 bp, synthetic construct; identical to SEQ ID NO: 81
  misc_feature 1..50: note = Haemophilus influenzae Gene Target Sequences Detected for Patients in Table 19 uppB 3209.446 seq_3
  source 1..50: mol_type = other DNA

SEQ ID NO: 103 - DNA, 50 bp, synthetic construct; identical to SEQ ID NO: 85
  misc_feature 1..50: note = Haemophilus influenzae Gene Target Sequences Detected for Patients in Table 19 vapD 9267.92 seq_5
  source 1..50: mol_type = other DNA

SEQ ID NO: 104 - DNA, 50 bp, synthetic construct; identical to SEQ ID NO: 33
  misc_feature 1..50: note = Pseudomonas aeruginosa Gene Target Sequences Detected for Patients in Table 19 dksA_1 19668.59 seq_9
  source 1..50: mol_type = other DNA

SEQ ID NO: 105 - DNA, 50 bp, synthetic construct; identical to SEQ ID NO: 52
  misc_feature 1..50: note = Pseudomonas aeruginosa Gene Target Sequences Detected for Patients in Table 19 fecI_11 188.791 seq_13
  source 1..50: mol_type = other DNA

SEQ ID NO: 106 - DNA, 50 bp, synthetic construct; identical to SEQ ID NO: 30
  misc_feature 1..50: note = Pseudomonas aeruginosa Gene Target Sequences Detected for Patients in Table 19 fecI_5 7826.244 seq_10
  source 1..50: mol_type = other DNA

SEQ ID NO: 107 - DNA, 50 bp, synthetic construct; identical to SEQ ID NO: 73
  misc_feature 1..50: note = Pseudomonas aeruginosa Gene Target Sequences Detected for Patients in Table 19 iscR 1098.42 seq_14
  source 1..50: mol_type = other DNA

SEQ ID NO: 108 - DNA, 50 bp, synthetic construct; identical to SEQ ID NO: 28
  misc_feature 1..50: note = Pseudomonas aeruginosa Gene Target Sequences Detected for Patients in Table 19 mucA 18647.4 seq_11
  source 1..50: mol_type = other DNA

SEQ ID NO: 109 - DNA, 50 bp, synthetic construct; identical to SEQ ID NO: 27
  misc_feature 1..50: note = Pseudomonas aeruginosa Gene Target Sequences Detected for Patients in Table 19 pslM 669.3498 seq_12
  source 1..50: mol_type = other DNA

SEQ ID NO: 110 - DNA, 50 bp, synthetic construct; identical to SEQ ID NO: 3
  misc_feature 1..50: note = Staphylococcus aureus Gene Target Sequences Detected for Patients in Table 19 rot 247.3 seq_16
  source 1..50: mol_type = other DNA

SEQ ID NO: 111 - DNA, 50 bp, synthetic construct; identical to SEQ ID NO: 2
  misc_feature 1..50: note = Staphylococcus aureus Gene Target Sequences Detected for Patients in Table 19 speH 432.3 seq_17
  source 1..50: mol_type = other DNA

SEQ ID NO: 112 - DNA, 50 bp, synthetic construct; identical to SEQ ID NO: 1
  misc_feature 1..50: note = Staphylococcus aureus Gene Target Sequences Detected for Patients in Table 19
  source 1..50: mol_type = other DNA

SEQ ID NO: 113 - DNA, 50 bp, synthetic construct
  misc_feature 1..50: note = Mycoplasma pneumoniae Gene Target Sequences Detected for Patients in Table 19 hisS 2730.578 seq_19
  source 1..50: mol_type = other DNA
tcttggtgttcttgatctttgtgcttttggagtaaagcattttccaagat

SEQ ID NO: 114 - DNA, 50 bp, synthetic construct
  misc_feature 1..50: note = Mycoplasma pneumoniae Gene Target Sequences Detected for Patients in Table 19 rpoD 2765.224 seq_20
  source 1..50: mol_type = other DNA
gcgagcactaccagtttagttgtgaagtgataggtgacactaatccaacc

SEQ ID NO: 115 - DNA, 50 bp, synthetic construct
  misc_feature 1..50: note = Mycoplasma pneumoniae Gene Target Sequences Detected for Patients in Table 19
  source 1..50: mol_type = other DNA
ttatcacgattagagatgtttttggtggttaattcttcaatgtcctcgtc

SEQ ID NO: 116 - DNA, 50 bp, synthetic construct; identical to SEQ ID NO: 86
  misc_feature 1..50: note = Coronavirus HKU1 Gene Target Sequences Detected for Patients in Table 19 Replicase 1B 918 seq_25
  source 1..50: mol_type = other DNA

SEQ ID NO: 117 - DNA, 50 bp, synthetic construct; identical to SEQ ID NO: 87
  misc_feature 1..50: note = Coronavirus HKU1 Gene Target Sequences Detected for Patients in Table 19 Spike 26 seq_26
  source 1..50: mol_type = other DNA

SEQ ID NO: 118 - skipped

SEQ ID NO: 119 - DNA, 50 bp, synthetic construct
  misc_feature 1..50: note = Hepatitis G 5 UTR Gene Target Sequences Detected for Patients in Table 19 5'UTR 397 seq_27
  source 1..50: mol_type = other DNA
agggttggtaggtcgtaaatcccggtcaccttggtagccactataggtgg

SEQ ID NO: 120 - DNA, 50 bp, synthetic construct; identical to SEQ ID NO: 53
  misc_feature 1..50: note = Candida albicans Gene Target Sequences Detected for Patients in Table 19 MEY_03361 434 seq_21
  source 1..50: mol_type = other DNA

SEQ ID NO: 121 - DNA, 50 bp, synthetic construct; identical to SEQ ID NO: 54
  misc_feature 1..50: note = Candida albicans Gene Target Sequences Detected for Patients in Table 19 MEY_04223 24 seq_22
  source 1..50: mol_type = other DNA

SEQ ID NO: 122 - DNA, 50 bp, synthetic construct
  misc_feature 1..50: note = Candida albicans Gene Target Sequences Detected for Patients in Table 19 MEY_06242 30 seq_23
  source 1..50: mol_type = other DNA
tatgttaatcctaagactatggcatttggccaaatttatcatggtgttca

SEQ ID NO: 123 - DNA, 50 bp, synthetic construct; identical to SEQ ID NO: 53
  misc_feature 1..50: note = Candida albicans Gene Target Sequences Detected for Patients in Table 19 MEY_03361 79 seq_21
  source 1..50: mol_type = other DNA

SEQ ID NO: 124 - DNA, 50 bp, synthetic construct; identical to SEQ ID NO: 54
  misc_feature 1..50: note = Candida albicans Gene Target Sequences Detected for Patients in Table 19 MEY_04223 127 seq_22
  source 1..50: mol_type = other DNA

SEQ ID NO: 125 - DNA, 50 bp, synthetic construct; identical to SEQ ID NO: 55
  misc_feature 1..50: note = Candida albicans Gene Target Sequences Detected for Patients in Table 19 MEY_05088 81 seq_24
  source 1..50: mol_type = other DNA

SEQ ID NO: 126 - DNA, 50 bp, synthetic construct; identical to SEQ ID NO: 122
  misc_feature 1..50: note = Candida albicans Gene Target Sequences Detected for Patients in Table 19 MEY_06242 132 seq_23
  source 1..50: mol_type = other DNA

SEQ ID NO: 127 - DNA, 50 bp, synthetic construct
  misc_feature 1..50: note = Sequences Detected for each AMR Gene Target for each Patient in Table 19 with a Bacterial Pathogen and at least one AMR Gene Detected aac(6')-Iak 38 seq_53
  source 1..50: mol_type = other DNA
cttccacgcgactgtccgaagccagttcgcggcagcccgcgtcgcgcgtc

SEQ ID NO: 128 - DNA, 44 bp, synthetic construct
  misc_feature 1..44: note = Sequences Detected for each AMR Gene Target for each Patient in Table 19 with a Bacterial Pathogen and at least one AMR Gene Detected blaPDC 121 seq_50
  source 1..44: mol_type = other DNA
cgcctacgtggcgttcgtcccgggccgcgacctgggactggtga

SEQ ID NO: 129 - DNA, 50 bp, synthetic construct
  misc_feature 1..50: note = Sequences Detected for each AMR Gene Target for each Patient in Table 19 with a Bacterial Pathogen and at least one AMR Gene Detected norA 444 seq_45
  source 1..50: mol_type = other DNA
tttttgctaatggctattggtcaataatgttaatcagttttgttgtcttc

SEQ ID NO: 130 - DNA, 50 bp, synthetic construct
  misc_feature 1..50: note = Sequences Detected for each AMR Gene Target for each Patient in Table 19 with a Bacterial Pathogen and at least one AMR Gene Detected erm(G) 49 seq_46
  source 1..50: mol_type = other DNA
gaaacttttgtaatgaaatgggttaacaaagagtatgaaaaactgtttac

SEQ ID NO: 131 - DNA, 50 bp, synthetic construct
  misc_feature 1..50: note = Sequences Detected for each AMR Gene Target for each Patient in Table 19 with a Bacterial Pathogen and at least one AMR Gene Detected sul2 66 seq_44
  source 1..50: mol_type = other DNA
gagtttggcagatgatttcgccaattgcggatagaacgcagcgtctggaa

SEQ ID NO: 132 - DNA, 50 bp, synthetic construct
  misc_feature 1..50: note = Sequences Detected for each AMR Gene Target for each Patient in Table 19 with a Bacterial Pathogen and at least one AMR Gene Detected aph(3'')-Ib 400 seq_42
  source 1..50: mol_type = other DNA
ccggtaagaagtcgggattgacggcattgcgggacaccacatcaacggcg

SEQ ID NO: 133 - DNA, 50 bp, synthetic construct
  misc_feature 1..50: note = Sequences Detected for each AMR Gene Target for each Patient in Table 19 with a Bacterial Pathogen and at least one AMR Gene Detected sul2 390 seq_43
  source 1..50: mol_type = other DNA
cgcctgtttcgtccgacacagaaatcgcgcgtatcgcgccggtgctggac

SEQ ID NO: 134 - DNA, 50 bp, synthetic construct; identical to SEQ ID NO: 132
  misc_feature 1..50: note = Sequences Detected for each AMR Gene Target for each Patient in Table 19 with a Bacterial Pathogen and at least one AMR Gene Detected aph(3'')-Ib 32 seq_42
  source 1..50: mol_type = other DNA

SEQ ID NO: 135 - DNA, 50 bp, synthetic construct; identical to SEQ ID NO: 131
  misc_feature 1..50: note = Sequences Detected for each AMR Gene Target for each Patient in Table 19 with a Bacterial Pathogen and at least one AMR Gene Detected sul2 78 seq_44
  source 1..50: mol_type = other DNA

SEQ ID NO: 136 - DNA, 50 bp, synthetic construct
  misc_feature 1..50: note = Sequences Detected for each AMR Gene Target for each Patient in Table 19 with a Bacterial Pathogen and at least one AMR Gene Detected aph(3'')-Ib 23 seq_47
  source 1..50: mol_type = other DNA
gcctcatttggctcaaaggtcgaggtgtggcttgccccgaggtgatcaac

(19) United States
(12) Patent Application Publication
(10) Pub. No.: US 2026/0110042 A1
(43) Pub. Date: Apr. 23, 2026

(54) METHODS FOR DETECTING INFECTIONS AND ASSOCIATED IMMUNE RESPONSES

(71) Applicants: PEAK DIAGNOSTICS PARTNERS INC., Aurora, CO (US); NATIONAL JEWISH HEALTH, Denver, CO (US)

(72) Inventors: Tasha FINGERLIN, Denver, CO (US); Roland MARCUS, Aurora, CO (US); Richard MARTIN, Aurora, CO (US); Brian O'CONNOR, Aurora, CO (US); Kendra WALTON, Aurora, CO (US)

(21) Appl. No.: 18/864,186

(22) PCT Filed: May 11, 2023
(86) PCT No.: PCT/US2023/021956

§ 371 Date: Nov. 8, 2024

Related U.S. Application Data
(60) Provisional application No. 63/340,831, filed on May 11, 2022.
(60) Provisional application No. 63/465,189, filed on May 9, 2023.

(51) Int. Cl. C12Q 1/70; C12Q 1/689; C12Q 1/6895

(52) U.S. Cl. CPC C12Q 1/701; C12Q 1/689; C12Q 1/6895; C12Q 2600/106; C12Q 2600/156

(57) ABSTRACT

A system for detecting infection is provided. The system includes a pathogen detection component for detecting a pathogen, a host immune response component for detecting a host immune response associated with the pathogen, and an antimicrobial resistance component for detecting an antimicrobial resistance associated with the pathogen.

### CROSS-REFERENCE TO RELATED APPLICATIONS

This application is a national stage application of PCT/US2023/021956, filed May 11, 2023, which claims priority to and the benefit of U.S. Provisional Patent Application Ser. No. 63/340,831, filed May 11, 2022, and entitled “TOOL FOR DETECTING INFECTIONS AND METHODS OF USING THE SAME”, and U.S. Provisional Patent Application Ser. No. 63/465,189, filed May 9, 2023, and entitled “METHODS FOR DETECTING INFECTIONS”, the disclosures of which are incorporated herein by reference in their entirety.

### FIELD

This disclosure is in the field of next-generation sequencing (NGS), and in particular in the field of utilizing NGS for detecting infections through the use of particular panels, as described herein.

### BACKGROUND

The diagnosis of pathogen infections has been developed over the past several decades. In the past, doctors and medical professionals relied mainly on physical symptoms and patient history to make a diagnosis. This approach, known as clinical diagnosis, was often imprecise and sometimes led to misdiagnosis or delayed diagnosis. With the advent of new diagnostic technologies and methods, the accuracy and speed of pathogen diagnosis have improved. Common methods for diagnosing pathogen infections is through laboratory testing. There are various laboratory methods available, including culture-based methods, serological tests, and molecular diagnostics.

Current methods for diagnosis of pathogen infections may require weeks to complete, have low sensitivity, and/or are limited in the scope of pathogens that can be detected. In the absence of a positive result, some physicians still prescribe antibiotics due to a fear of undertreating patients. Other physicians, aware of over-use, do not prescribe antibiotics even though an infection may exist. Therefore, improved tools and methods for diagnosing infections and determining appropriate treatment plans are needed.

### SUMMARY

In an aspect, a system for detecting infection is disclosed. The system includes a pathogen detection component for detecting a pathogen; a host immune response component for detecting a host immune response associated with the pathogen; and an antimicrobial resistance component for detecting an antimicrobial resistance associated with the pathogen.

In embodiments, the pathogen detection component detects mRNA for species-specific detection of one or more pathogens selected from the group consisting of bacterial pathogens, viral pathogens, and fungal pathogens. In embodiments, the host immune response component detects a human immune gene expression profile. In embodiments, the antimicrobial resistance component detects anti-microbial resistance gene expression.

In another aspect, a panel for detecting a pathogen and an associated biological response in an ex vivo biological sample is disclosed. The panel includes a pathogen detection component for amplifying at least a portion of a gene associated with the pathogen; and an immune-response component for amplifying at least a portion of a gene associated with an immune response associated with the pathogen.

In embodiments, the panel further includes an antimicrobial resistance component for amplifying at least a portion of a gene associated with an antimicrobial resistance to the pathogen.

In embodiments, the pathogen is a bacteria, a virus, or a fungus. In embodiments, the bacteria is Staphylococcus aureus, Pseudomonas aeruginosa, Haemophilus parainfluenzae, Haemophilus influenzae, Mycoplasma pneumoniae, or a mutant thereof. In embodiments, the fungus is Candida albicans, Aspergillus fumigatus, Candida glabrata, or a mutant thereof. In embodiments, the virus is a Hepatitis virus or a Coronavirus, or a mutant thereof.

In embodiments, when the bacteria is Staphylococcus aureus, the gene is selected from one or more of dksA_1, fec1_11, fec1_12, fec1_5, iscR, mucA, psIM, pTIF-4, speH, rot, femC, and IpI3_1. In embodiments, when the bacteria is Pseudomonas aeruginosa, the gene is selected from one or more of psiM, mucA, iscR, fec1_5, fec1_12, fec1_11, ptIF_4, and dksA_1. In embodiments, when the bacteria is Haemophilus parainfluenzae, the gene is selected from one or more of 16s_1. 16s_2, and 16s_3. In embodiments, when the bacteria is Haemophilus influenzae, the gene is selected from one or more of hgpC, relB, uppB, vapD, and yafQ_1. In embodiments, when the bacteria is Mycoplasma pneumoniae, and the gene is selected from one or more of galU, hisS, and rpoD. In embodiments, when the fungus is Candida albicans, the gene is selected from one or more of MEY_03361, MEY_04223, MEY_05088, and MEY_06242. In embodiments, when the fungus is Aspergillus fumigatus, and the gene is selected from one or more of AFUA_1G05430, AFUA_8G00910, and AFUA_8G02500. In embodiments, when the fungus is Candida glabrata, and the gene is selected from one or more of CAGLOA00187g, CAGLOA00297g, and CAGLOA00803g. In embodiments, when the virus is Coronavirus HKU1, the gene is selected from one or more of Replicase 1B and Spike. In embodiments, when the virus is Hepatitis G, the gene is 5′UTR.

In embodiments, the gene associated with an antimicrobial resistance is selected from one or more of aph(3″)=1b, aac(6′)-lak, blaPDC, erm(G), norA, and sul2.

In embodiments, the sequence of the gene is at least 80%, or at least 90%, or at least 95% identical to any one of SEQ ID NOs: 1-26, or 110-112. In embodiments, the sequence of the gene is at least 80%, or at least 90%, or at least 95% identical to any one of SEQ ID NOs: 27-52, 63-75, or 104-109. In embodiments, the sequence of the gene is at least 80%, or at least 90%, or at least 95% identical to any one of SEQ ID NOs: 53-56, or 120-126. In embodiments, the sequence of the gene is at least 80%, or at least 90%, or at least 95% identical to any one of SEQ ID NOS: 57-59. In embodiments, the sequence of the gene is at least 80%, or at least 90%, or at least 95% identical to any one of SEQ ID NOs: 60-62. In embodiments, the sequence of the gene is at least 80%, or at least 90%, or at least 95% identical to any one of SEQ ID NOs: 76-78. In embodiments, the sequence of the gene is at least 80%, or at least 90%, or at least 95% identical to any one of SEQ ID NOs: 79-85. In embodiments, the sequence of the gene is at least 80%, or at least 90%, or at least 95% identical to any one of SEQ ID NOs: 86-87, or 116-118. In embodiments, the sequence of the gene is at least 80%, or at least 90%, or at least 95% identical to any one of SEQ ID NOS: 88-103. In embodiments, the sequence of the gene is at least 80%, or at least 90%, or at least 95% identical to any one of SEQ ID NOs: 113-115. In embodiments, the sequence of the gene is at least 80%, or at least 90%, or at least 95% identical to SEQ ID NO: 119.

In embodiments, the immune-response component is associated with a bacterial pathogen, a viral pathogen, or a fungal pathogen. In embodiments, the immune-response component is associated with a bacterial pathogen, and the gene is selected from TRAF3, PIK3CD, RUNX1, IFNB1, IFNA5, RORC, IFNG-AS1, IFNA4, IFNA2, and RORA. In embodiments, the immune-response component is associated with a bacterial pathogen, and the gene is selected from VAVI, PRKCD, ICOSLG, IRAK2, TICAM1, CSF3, TRAF3, AGER, PTGER4, and BCL6. In embodiments, the immune-response component is associated with a viral pathogen, and the gene is selected from SOCS1, IL13, CSF2, PRKCD, NLRP3, DDX3X, CCR8, TGFB1, CCL17, and TNFRSF4. In embodiments, the sequence of the gene is at least 80%, or at least 90%, or at least 95% identical to any one of SEQ ID NOs: 127-136.

### BRIEF DESCRIPTION OF THE FIGURES

FIG. 1 illustrates an algorithm for predicting infection in accordance with embodiments of the disclosure.

FIG. 2 illustrates a limit of detection assay in accordance with embodiments of the disclosure.

### DETAILED DESCRIPTION

### Overview of the Detailed Description

The detailed description of exemplary embodiments references the accompanying drawing figures, which show exemplary embodiments by way of illustration and their best mode. While these exemplary embodiments are described in sufficient detail to enable those persons skilled in the art to practice the invention, it should be understood that other embodiments may be realized and that logical, chemical, and mechanical changes may be made without departing from the spirit and scope of the inventions detailed herein. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

A sequencing diagnostic panel is a type of molecular diagnostic test that uses next-generation sequencing (NGS) technology to detect and identify pathogens in patient samples. Disclosed herein is a comprehensive, yet targeted, next-generation sequencing (NGS) diagnostic panel for identifying infections.

### Definitions and Interpretation

If and as used herein, the term “pathogen detection” refers to a component of: (1) six (6) genes, each of which provides speciation information across a wide range of microbes, such that the combined information enables global detection of potential pathogens; and (2) multiple mRNAs for species-specific detection of bacterial, viral, and fungal pathogens.

If and as used herein, the term “anti-microbial resistance” refers to a component that detects anti-microbial resistance (AMR) gene expression.

If and as used herein, the term “host immune gene expression” component measures the human immune/inflammatory profile. Results from all components of the panel are derived from the same patient sample in the same assay.

### Overview of Aspects & Embodiments

In an aspect, a system for detecting infection is disclosed. The system includes a pathogen detection component for detecting a pathogen; a host immune response component for detecting a host immune response associated with the pathogen; and an antimicrobial resistance component for detecting an antimicrobial resistance associated with the pathogen.

In embodiments, the pathogen detection component detects mRNA for species-specific detection of one or more pathogens selected from the group consisting of bacterial pathogens, viral pathogens, and fungal pathogens. In embodiments, the host immune response component detects a human immune gene expression profile. In embodiments, the antimicrobial resistance component detects anti-microbial resistance gene expression.

In another aspect, a panel for detecting a pathogen and an associated biological response in an ex vivo biological sample is disclosed. The panel includes a pathogen detection component for amplifying at least a portion of a gene associated with the pathogen; and an immune-response component for amplifying at least a portion of a gene associated with an immune response associated with the pathogen.

In embodiments, the panel further includes an antimicrobial resistance component for amplifying at least a portion of a gene associated with an antimicrobial resistance to the pathogen.

In embodiments, the pathogen is a bacteria, a virus, or a fungus. In embodiments, the bacteria is Staphylococcus aureus, Pseudomonas aeruginosa, Haemophilus parainfluenzae, Haemophilus influenzae, Mycoplasma pneumoniae, or a mutant thereof. In embodiments, the fungus is Candida albicans. Aspergillus fumigatus, Candida glabrata, or a mutant thereof. In embodiments, the virus is a Hepatitis virus or a Coronavirus, or a mutant thereof.

In embodiments, when the bacteria is Staphylococcus aureus, the gene is selected from one or more of dksA_1, fec1_11, fec1_12, fec1_5, iscR, mucA, psIM, pTIF-4, speH, rot, femC, and IpI3_1. In embodiments, when the bacteria is Pseudomonas aeruginosa, the gene is selected from one or more of psiM, mucA, iscR, fec1_5, fec1_12, fec1_11. ptIF_4, and dksA_1. In embodiments, when the bacteria is Haemophilus parainfluenzae, the gene is selected from one or more of 16s_1. 16s_2, and 16s_3. In embodiments, when the bacteria is Haemophilus influenzae, the gene is selected from one or more of hgpC, relB, uppB, vapD, and yafQ_1. In embodiments, when the bacteria is Mycoplasma pneumoniae, and the gene is selected from one or more of galU, hisS, and rpoD. In embodiments, when the fungus is Candida albicans, the gene is selected from one or more of MEY_03361, MEY_04223, MEY_05088, and MEY_06242. In embodiments, when the fungus is Aspergillus fumigatus, and the gene is selected from one or more of AFUA_1G05430, AFUA_8G00910, and AFUA_8G02500. In embodiments, when the fungus is Candida glabrata, and the gene is selected from one or more of CAGLOA00187g, CAGLOA00297g, and CAGLOA00803g. In embodiments, when the virus is Coronavirus HKU1, the gene is selected from one or more of Replicase 1B and Spike. In embodiments, when the virus is Hepatitis G, the gene is 5′UTR.

In embodiments, the gene associated with an antimicrobial resistance is selected from one or more of aph(3″)=lb, aac(6′)-lak, blaPDC, erm(G), norA, and sul2.

In embodiments, the sequence of the gene is at least 80%, or at least 90%, or at least 95% identical to any one of SEQ ID NOs: 1-26, or 110-112. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 27-52, 63-75, or 104-109. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 53-56, or 120-126. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 57-59. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 60-62. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 76-78. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 79-85. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 86-87, or 116-118. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 88-103. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 113-115. In embodiments, the sequence of the gene is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 119.

In embodiments, the immune-response component is associated with a bacterial pathogen, a viral pathogen, or a fungal pathogen. In embodiments, the immune-response component is associated with a bacterial pathogen, and the gene is selected from TRAF3, PIK3CD, RUNX1, IFNB1, IFNA5, RORC, IFNG-AS1, IFNA4, IFNA2, and RORA. In embodiments, the immune-response component is associated with a bacterial pathogen, and the gene is selected from VAVI, PRKCD, ICOSLG, IRAK2, TICAM1, CSF3, TRAF3, AGER, PTGER4, and BCL6. In embodiments, the immune-response component is associated with a viral pathogen, and the gene is selected from SOCS1, IL13, CSF2, PRKCD, NLRP3, DDX3X, CCR8, TGFB1, CCL17, and TNFRSF4. In embodiments, the sequence of the gene is at least 80%, or at least 90%, or at least 95% identical to any one of SEQ ID NOs: 127-136.

Results from this system and the associated panel can be used to effectively treat an individual based on the appropriate detection of the pathogen. Notably, the disclosed system and panel allow for the rapid and correct determination of a pathogen based on pathogen detection, host immune gene expression, and anti-microbial resistance phenomena.

### Detailed Description of Embodiments

A sequencing diagnostic panel is a type of molecular diagnostic test that uses next-generation sequencing (NGS) technology to detect and identify pathogens in patient samples. Unlike traditional diagnostic tests, which are often limited to detecting one or a few specific pathogens, sequencing diagnostic panels can simultaneously screen for hundreds or thousands of different pathogens in a single test.

The process of using a sequencing diagnostic panel typically involves collecting a sample from the patient, such as blood, urine, or tissue, and extracting the genetic material from the sample. The extracted genetic material is then processed and sequenced using NGS technology. The resulting data is analyzed using bioinformatics software, which compares the genetic material to a reference database of known pathogens to identify any matches.

Sequencing diagnostic panels have several advantages over traditional diagnostic tests. For example, they can detect a wide range of pathogens, including bacteria, viruses, fungi, and parasites, in a single test. This can be especially useful for identifying rare or emerging pathogens that may not be included in traditional diagnostic panels. Additionally, sequencing diagnostic panels can provide a more accurate diagnosis by detecting genetic variations that may be missed by other tests. However, sequencing diagnostic panels can also be more expensive and time-consuming than traditional diagnostic tests, and may require specialized equipment and expertise to perform and interpret.

Disclosed herein is a comprehensive, yet targeted, next-generation sequencing (NGS) diagnostic panel for identifying infections, such as microbial pulmonary infections that result in poor symptom control of asthma, COPD and other chronic lung diseases. The innovative design of the panel permits screening for tens of thousands of microbial species and other pathogens in a single assay. The panel also detects anti-microbial resistance genes and human host response genes, enabling characterization of pathogenic activity, microbial longitudinal evolution, and the accuracy of pathogen versus commensal microbial identification. The increased sensitivity of the panel may be helpful for detection of various infection types and severity, such that the panel may be a valuable tool for clinical studies of antimicrobial compounds. The panel may also allow researchers to evaluate new treatment paradigms for syndromes, such as chronic lung disease, that target the immune system by studying the interplay between host and microbiome (both pathogenic and commensal) response over time. This application may be of particular value given the likely increased use of biologics to treat chronic lung disease, which can increase the risk of opportunistic infections. Used longitudinally, the panel may assess host and pathogen treatment response simultaneously, thus enabling monitoring for robust investigation of complex infections. Integrating host response with pathogen detection and antimicrobial resistance data may provide evidence of pathogenic microbes, and help measure response to treatment, shifting microbial activity and/or anti-microbial-resistance evolution.

The panel may identify pathogens in samples with a low burden of nucleic acids compared to the amount of human nucleic acids. This may occur with less severe infection setting, or in certain other human clinical sample types, such as, for example, synovial fluid, in the context of an acute infection. The panel may use samples directly from the patient, such as bronchoalveolar lavage fluid, sputum, synovial fluid, or tissue biopsy. The panel may screen for hundreds, thousands or hundreds of thousands of bacterial, fungal, and viral species in a single assay by sequencing nucleic acids from genes common to pathogens (e.g., microbial 16s, 18s and ITS (internal transcribed spacer)), but that have variable regions that distinguish among different species.

The NGS panel consists of three major components. The “Pathogen Detection” component is based on 1) six (6) genes, each of which provides speciation information across a wide range of microbes, such that the combined information enables global detection of potential pathogens, and 2) multiple species-specific mRNAs for species-specific detection of bacterial, viral, and fungal pathogens. The bacterial detection includes gram stain negative (e.g., Haemophilus influenzae) and gram stain positive species (e.g., Staphylococcus aureus), mollicute species (e.g., Mycoplasma pneumoniae), other atypical (do not get colored by gram staining) species (e.g., Chlamydophila pneumoniae), mycobacterial species (tuberculous and non-tuberculous). The fungal detection includes yeast (e.g., Candida albicans) and molds (e.g., Aspergillus fumigatus). The viral detection includes single stranded RNA (e.g., Coronavirus HKU1) double stranded RNA viruses (e.g., Rotavirus), single stranded DNA (e.g., Adeno-associated virus) and double stranded DNA viruses (e.g. Cytomegalovirus). The “Anti-Microbial Resistance” component detects anti-microbial resistance (AMR) gene expression for classes of anti-microbial compounds including the classes aminoglycoside, beta-lactam, fluoroquinolone, macrolide and sulfonamide. The “Host Immune Gene Expression” component measures human immune/inflammatory profiles.

The NGS panel provides a significant advantage over current testing methodology, as outlined in Table 1 herein.

TABLE 1
Comparison of characteristics of existing technologies

| | Pathogen | Comprehensive | Anti- | Human | Sequence- |
|---|---|---|---|---|---|
| | RNA (Activity | Species | Microbial | Immune | Based |
| Tech | Vs. Remnant | Capability | Resistance | Response | Speciation |
| Peak NGS Dx | ✓ | ✓ | ✓ | ✓ | ✓ |
| Culture | N/A | X | ~ | X | X |
| qPCR | ✓ | X | X | X | X |
| Metagenomics | X | ✓ | ~ | X | ✓ |
| Immune Profile | X | X | X | ✓ | X |

The majority of microbiological clinical testing does not use molecular methods, but instead relies on decades-old approaches for identification of pathogens based on cultures. These culture methods are limited to diagnosis of pathogens that can be grown in the laboratory with known conditions. Furthermore, the microbes that survive the sampling and culturing process may not represent the dominant species or strain driving the infection.

Some testing employs PCR-based methods. Many PCR assays still use samples from a culture, but there are point-of-care molecular diagnostic assays that use samples directly from a patient, including those from Biofire (https://www.biofiredx.com) and Cepheid (https://www.cepheid.com). These rapid point-of-care implementations of PCR have limits of detection (LoDs) that are substantially higher than PCR is capable of achieving when implemented in a reference laboratory. Even if appropriate LoDs for infections could be achieved with PCR, the challenges for any version of PCR for diagnosis are: (1) the low number of pathogens that can be targeted in a single assay (and the low number of targets per pathogen that can be included); and (2) the inability to know whether or not the amplified target truly came from the pathogen of interest. This second issue of specificity is particularly relevant because, since PCR does not provide sequence information, there is no way to check whether the detected sequence is from the targeted pathogen or other material. The NGS panel disclosed herein uses sequencing data to confirm an appropriate match between the detected nucleic acid and the pathogen.

Another previously used testing platform utilizes metagenomic assays. Metagenomic assays that sequence all of the DNA in a sample are attractive because they do not require a priori information about which pathogen(s) to test for and they have the potential to provide a survey of all of the organisms in the sample. However, these methods are generally less sensitive than targeted amplification methods because of the overwhelming amounts of human nucleic acid in the samples compared to the minute amounts of microbe nucleic acid. Because of this lack of sensitivity, current implementations are not considered appropriate for first-line clinical testing. In addition, the data analysis required for meta-genomics approaches is both complex and difficult to optimize. The NGS panel disclosed herein targets the microbe and human genes of interest, allowing both a broad coverage of pathogens and capture of the relevant human expression. This results in increased sensitivity for pathogen detection and simultaneous characterization of the immune response.

Another diagnostic approach uses human immune gene expression to predict whether an individual has a viral or bacterial infection or a combination of both. In this approach, dozens of genes for each algorithm are used to predict the type of infection. However, there is no actual detection of the pathogen and therefore no ability to definitively pair the immune response with a pathogen. This approach also requires that testing be done in blood samples.

CRISPR-based technologies are being developed for diagnostics of infectious disease and may be cheaper on a per-assay basis, but they do not have higher sensitivity than current culture or PCR techniques, and are not yet able to be highly multiplexed limiting their comparability to the presently disclosed sensitive and comprehensive panel, which also includes detection of the host immune response.

As described above, and detailed below, the NGS panel disclosed herein is a significant improvement over currently employed methods. The panel is designed for use on either the Thermo Ion Next Generation Sequencing (NGS) platform (https://www.thermofisher.com/order/catalog/product/A27212) or the Illumina Next Generation Sequencing platform (https://www.illumina.com). However, any NGS sequencing platform may be used.

### EXAMPLES

### Example 1. Limit of Detection Assay

### Sample Collection

Staphylococcus aureus (also referred to herein as S. aureus or “staph”) has long been recognized as a bacteria that causes disease in humans. It is the leading cause of skin and soft tissue infections such as abscesses (boils), furuncles, and cellulitis. Although most staph infections are not serious, S. aureus can cause serious infections such as bloodstream infections, pneumonia, or bone and joint infections.

Pseudomonas is a type of bacteria that is commonly found in the environment, such as in soil or water. Of the many different types of Pseudomonas, the one that most often causes infections in humans is Pseudomonas aeruginosa, which can cause infections in the blood, lungs (pneumonia), or other parts of the body after surgery.

In this Example, simultaneous detections of both bacterial species were carried out.

### Collection of Samples

Staphylococcus aureus and Pseudomonas aeruginosa were grown from glycerol stocks at 37° C.; S. aureus in tryptic soy broth (TSB) and P. aeruginosa in lysogeny broth (LB). S. aureus and P. aeruginosa were mixed into one tube at approximately even bacterial numbers (1×10⁷ of each species). Ten-fold serial dilutions were performed taking 67 μl from the original mixed tube using 600 μl 1× Shield in each dilution. 1:10 serial dilutions were made to obtain 10⁷ to 10⁰ dilutions. 10^{X} references the estimated number of organisms in the dilution based on ocular density; estimated number of organisms decreases from 10⁷ to 10⁰. Once samples were diluted, 6 μl of human sputum was added to every dilution so that the human sputum stayed constant as the bacterial strains were diluted out in 1× Shield. In a concurrent experiment, the same serial dilutions were made in PBS, removing the 1× Shield as a buffer, so that this dilution series could be plated on an appropriate medium for S. aureus and P. aeruginosa growth. For each of the 8 dilutions, 20 μl of the PBS plus bacterial mixture was plated on 3 plates; one with specific growth factors for Pseudomonas, one for Staphylococcus and one non-specific LB plate. Each pathogen was considered detected at a given dilution point if there were at least 3 colony forming units (CFU) observed on the plate, a liberal criteria compared to usual clinical testing (see, for e.g.: O'Toole et al. (2016) J. Bacteriol.). P. aeruginosa was detected at the 10⁷, 10⁶, 10⁵, and 10⁴ dilutions, but not detected at lower dilution points; therefore, the LoD based on culture was 10⁴ for P. aeruginosa. S. aureus was detected at the 10⁷, 10⁶, 10⁵, 10⁴, 10³ dilutions, but not detected at lower dilution points; therefore, the LoD based on culture was 10³ for S. aureus.

### Extraction of Nucleic Acid

Samples were thawed on ice and vortexed to mix well. 2 ml screw top tubes were filled ˜⅓ full with 1 mm Zirconia/Silica beads. After samples were transferred to the 2 ml screw top tubes they were placed in a MP Bio FastPrep-24 classic bead beating grinder and lysis system. The samples were bead beat at 6.5 m/s for 45s for 2 rounds. The samples were centrifuged at >8,000g for 5 min to pellet the beads and the supernatant for each sample was transferred to a new 1.7 ml tube. The samples were treated with ProK and incubated at room temperature (RT) for 30 mins. Using the Zymo Quick DNA/RNA Miniprep kit, following the DNA and RNA purification protocol, 1 volume of Lysis buffer was added. The entire sample was transferred to the Spin-Away Filter and centrifuged at >8,000g for 30s. The Spin-Away Filter was transferred to a new collection tube while the RNA was processed from the flow through. One volume of ethanol was added to the RNA flow through and transferred to a Zymo-Spin IICR column and centrifuged at full speed for 30s. DNase treatment was performed on all samples, followed by a DNA/RNA Prep Buffer wash and two DNA/RNA washes, and eluted in 50 μl of DNase/RNase free water. The DNA was then extracted by adding DNA/RNA Prep Buffer to the Spin-Away Filter, followed with two rounds of washes. The samples were eluted in 50 μl DNase/RNase free water.

### Sequencing Library Preparation

Ion Torrent libraries were prepared using a Thermo Fisher custom assay and the Ion Torrent Library Plus kit and protocol (Thermo Fisher. Accessed Mar. 28, 2022, https://www.thermofisher.com/document-connect/document-connect.html?url=https % 3A %2F %2Fassets.thermofisher.com %2FTFS-Assets % 2FLSG %2Fmanuals %2FMAN0017003_IonAmpliSeqLibaryKitPlus_UG.pdf).

RNA from mixed pathogen sputum dilution series samples (100 ng) was reverse transcribed using SuperScript IV VILO Master Mix. For each cDNA sample, PCR master mix was added to 10 μl of cDNA. Each sample was amplified for 16-20 cycles with a 4-16 minute anneal/extend time according to protocol. After digestion of the primer sequences, barcoded adapters were added to each sample, each sample was cleaned using Ampure XP beads, and the beads were eluted with a library amplification master mix. Libraries underwent a brief PCR, were cleaned with a double Ampure XP bead clean up eluted in 50 μl Low TE. Libraries were quantified and pooled based on concentration to obtain equal representation of each sample. Each pool was templated on the Ion Chef Instrument and sequenced on the Ion GeneStudio™ S5 Prime System.

### NGS Pathogen Gene Detection Analysis

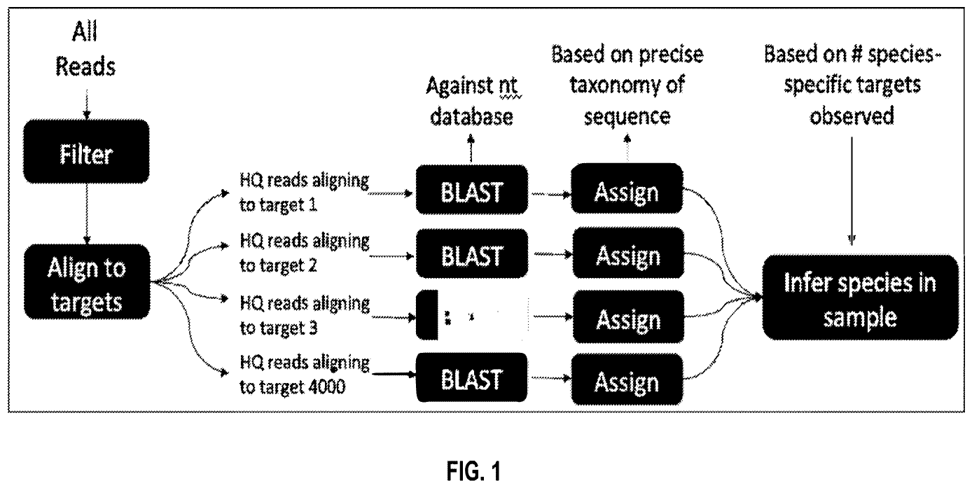

To determine whether and which bacterial, fungal and/or viral species are present in a given sample based on the NGS panel, the algorithm depicted in FIG. 1 was applied to the raw sequencing data based on the library for that sample. Specifically, with respect to FIG. 1, the raw sequencing files were filtered to keep reads that were at least 100 bp long. Reads were aligned to the database of expected sequences based on the NGS panel design using Bowtie2 with “very-sensitive setting (see, for e.g.: Langmead et al. “Scaling read aligners to hundreds of threads on general-purpose processors.” Bioninformatics). Reads that aligned with Bowtie2 were considered potential matches to a target gene sequence in the NGS panel design (target), and those read sequences were then compared to the nucleotide nt database (Nucleotide [Internet]. Bethesda (MD): National Library of Medicine (US) NCBI) to determine the single or multiple species consistent with the read's sequence, noting the % identity of each mapping event. Detection of a bacterial or fungal species in a sample required at least 3 gene targets of that species present in that sample. Detection of a viral species in a sample required at least one gene target of that species be detected in the sample given the much smaller genome sizes. For all pathogens, for a target to be detected for a given species, the reads for that target were required to have >97% identity to the intended species and no greater than 97% identity to any sequence in the database for any other species. The number of reads supporting each target was required to be at least 20 CPM to be considered detected above background. These criteria enhance specificity by requiring a close match of the detected sequence to a specific species with no alternative species being consistent with the target as is described next.

Tables 2 and 3 display, for each of S. aureus and P. aeruginosa at each dilution point, 4 and 8 of the gene targets that were detected for that pathogen, respectively. S. aureus was detected by the NGS panel at all dilution points but 10¹ based on at least 3 gene targets being detected at each dilution point (Table 2). Since S. aureus was not detected at 10¹, the NGS panel LoD for S. aureus was 10² even though the panel did detect it at 10⁰. P. aeruginosa was detected by the NGS panel in dilutions 10⁷ through 10², but not at dilutions 10¹ and 10⁰ based on the requirement that at least 3 gene targets are detected (Table 3); dilutions 10¹ and 10⁰ had no gene targets detected. Hence, the NGS panel LoD for P. aeruginosa was also 10². No other pathogens were detected in any of the dilution series samples such that specificity was 100% across the entire dilution series for both pathogens. The data in Tables 2 and 3 also demonstrate the utility of having more than one gene target available for detection in the panel to increase sensitivity while maintaining specificity; different dilution points can have different genes detected vs. not detected. For example, as shown in Table 2, dilutions 10⁶-10² all had the same 4 genes detected (femC, IpI3_1, rot, speH), while the 10⁷ dilution had a subset of 3 of those 4 genes (femC, rot, speH) and 10° had a different subset of 3 genes (Ip13_1, rot, speH). Similarly, as shown in Table 3 for P. aeruginosa, in dilutions 10⁴ and 10⁵, P. aeruginosa was detected based on two of the same genes (mucA and psIM) in each, but 10⁴ had the fec1_12 gene as the third detected target whereas 10⁵ had the iscR gene as the third detected target. Dilutions 10³ (dksA_1, fec1_12, mucA) and 10² (fecI_11, fec1_5, ptIF_4) had other combinations of three genes detected. These three dilution points show that by having more than three potential targets in the panel for each pathogen, the sensitivity of the panel is robust to biological differences (e.g., different genes included in different strains of a pathogen genome) or sampling-derived differences (e.g., when taking a sample, certain nucleic acids may not be included in the sample) between samples. At the same time, by requiring three different targets, each with uniquely high sequence similarity to the pathogen, the panel demonstrates specificity (low false positive rate) for pathogen calls.

TABLE 2
Staphylococcus aureus Genes Detected at each Dilution Point

Species
Staphylococcus aureus*
Gene

| Dilution | femC | lpl3_1 | rot | speH |
|---|---|---|---|---|
| 10⁷ | DET | ND | DET | DET |
| 10⁶ | DET | DET | DET | DET |
| 10⁵ | DET | DET | DET | DET |
| 10⁴ | DET | DET | DET | DET |
| 10³ | DET | DET | DET | DET |
| 10² | DET | DET | DET | DET |
| 10¹ | ND | ND | ND | ND |
| 10⁰ | ND | DET | DET | DET |

*With reference to Table 2, “DET” means detected; and “ND” means not detected. 10^{X} references the estimated number of organisms in the dilution based on ocular density; estimated number of organisms decreases from 10⁷ to 10⁰.

TABLE 3
Pseudomonas aeruginosa Genes Detected at each Dilution Point

| | Species | | | | | | | |
|---|---|---|---|---|---|---|---|---|
| | Pseudomonas aeruginosa* | | | | | | | |
| | Gene | | | | | | | |
| Dilution | dksA_1 | fecI_11 | fecI_12 | fecI_5 | iscR | mucA | pslM | ptlF_4 |
| 10⁷ | DET | DET | DET | DET | DET | DET | DET | ND |
| 10⁶ | DET | DET | DET | DET | DET | DET | DET | ND |
| 10⁵ | ND | ND | ND | ND | DET | DET | DET | ND |
| 10⁴ | ND | ND | DET | ND | ND | DET | DET | ND |
| 10³ | DET | ND | DET | ND | ND | DET | ND | ND |
| 10² | ND | DET | ND | DET | ND | ND | ND | DET |
| 10¹ | ND | ND | ND | ND | ND | ND | ND | ND |
| 10⁰ | ND | ND | ND | ND | ND | ND | ND | ND |

*With reference to Table 3, “DET” means detected; and “ND” means not detected. 10^{X} references the estimated number of organisms in the dilution based on ocular density; estimated number of organisms decreases from 10⁷ to 10⁰.

We used DNA from an ATCC mock bacterial community (ATCC MSA-1002) to determine the analysis pipeline thresholds for determination of whether or not a given species was detected in a sample based on the NGS reads. The mock community included 8 pathogens targeted by our panel (Staphylococcus aureus, Pseudomonas aeruginosa, Escherichia coli, Cutibacterium acnes, Acinetobacter baumannii, Streptococcus agalactiae, Enterococcus faecalis, Staphylococcus epidermis). The samples were prepared as in Example 1 for library preparation, sequencing, and pathogen detection analysis, where the pathogen detection analysis was modified as described below to investigate a range for 1) the number of reads (in counts per million [CPM]) per target observed that are necessary to determine that the target is detected and 2) the number of targets per species that are necessary to determine that the species is detected. We considered combinations of between 5 CPM and 50 CPM as a threshold for target detection and between 1 and 10 targets detected per species as a threshold for species detection. We compared the sensitivity (probability of calling a species as detected when present in the sample) and number of false positive species detected at each combination of CPM and number of targets required. The optimal trade-off between sensitivity and number of false positive detections was identified as either of the following: A) 20 CPM with 2 or 3 targets per species or B) 15 CPM with 3 targets per species required. The sensitivity of the combination of 20 CPM and 3 targets required for bacterial or fungal species detection was 87.5% (7 of 8 detected) with zero false positive findings. No other combination had higher sensitivity without introducing false positive results.

For each of the genes and at each dilution point, Tables 4 and 5 show the sequence of the gene target from the sequencing data that was detected, the percentage of sequence similarity of the sequence to the reference pathogen sequence in the nt database (% identity), and the CPM for the detected gene target for S. aureus and P. aeruginosa, respectively. For each of the genes, the sequence similarity to the reference pathogen sequence is 100% with the exception of one gene that has 99.5% sequence identity. Importantly, even for that gene with 99.5% sequence identity, no other species had a reference sequence with higher than 90% sequence identity to the sequence we identified. These sequence similarities are key for the specificity of the panel performance; target sequences must be identified with very high sequence similarity to a reference sequence that is unique to the pathogen in order to determine that a pathogen is detected, which increases specificity and allows exclusion of pathogens with sequences less similar to the sequence identified in a sample.

TABLE 4
Sequences Detected for each Gene Target for Staphylococcus
aureus in Dilution Series

| | | | SEQ | % | |
|---|---|---|---|---|---|
| Dilution | Gene | CPM | Name | Identity | Sequence |
| 10⁷ | speH | 6070 | seq_ | 100 | AATATTTAGTTAACTCATTAACGTTAGTTTCGTTTT |
| | | | 17 | | GATTTATATAATAA (SEQ ID NO: 1) |
| | rot | 67299 | seq_ | 100 | TTAGAAACACTTTTGGGTGACATTAACTCAATTTT |
| | | | 16 | | CAGCGAGATTGAAAG (SEQ ID NO: 2) |
| | femC | 521517 | seq_ | 100 | TTCATCTGTTGTTAAATGCTCTTGTGAGTCATAAA |
| | | | 15 | | TGATTTGTTTAATCC (SEQ ID NO: 3) |
| 10⁶ | speH | 9087 | seq_ | 100 | AATATTTAGTTAACTCATTAACGTTAGTTTCGTTTT |
| | | | 17 | | GATTTATATAATAA (SEQ ID NO: 4) |
| | rot | 74752 | seq_ | 100 | TTAGAAACACTTTTGGGTGACATTAACTCAATTTT |
| | | | 16 | | CAGCGAGATTGAAAG (SEQ ID NO: 5) |
| | Ipl3_1 | 99 | seq | 100 | ATCACTTTTGCCACAACCTGCTATCGCAAATATTA |
| | | | 34 | | AAATCATA (SEQ ID NO: 6) |
| | femC | 457053 | seq | 100 | TTCATCTGTTGTTAAATGCTCTTGTGAGTCATAAA |
| | | | 15 | | TGATTTGTTTAATCC (SEQ ID NO: 7) |
| 10⁵ | speH | 3441 | seq_ | 100 | AATATTTAGTTAACTCATTAACGTTAGTTTCGTTTT |
| | | | 17 | | GATTTATATAATAA (SEQ ID NO: 8) |
| | rot | 66226 | seq_ | 100 | TTAGAAACACTTTTGGGTGACATTAACTCAATTTT |
| | | | 16 | | CAGCGAGATTGAAAG (SEQ ID NO: 9) |
| | Ipl3_1 | 240 | seq_ | 100 | ATCACTTTTGCCACAACCTGCTATCGCAAATATTA |
| | | | 34 | | AAATCATA (SEQ ID NO: 10) |
| | femC | 482885 | seq | 100 | TTCATCTGTTGTTAAATGCTCTTGTGAGTCATAAA |
| | | | 15 | | TGATTTGTTTAATCC (SEQ ID NO: 11) |
| 10⁴ | speH | 16980 | seq | 100 | AATATTTAGTTAACTCATTAACGTTAGTTTCGTTTT |
| | | | 17 | | GATTTATATAATAA (SEQ ID NO: 12) |
| | rot | 56168 | seq | 100 | TTAGAAACACTTTTGGGTGACATTAACTCAATTTT |
| | | | 16 | | CAGCGAGATTGAAAG (SEQ ID NO: 13) |
| | Ipl3_1 | 9888 | seq_ | 100 | ATCACTTTTGCCACAACCTGCTATCGCAAATATTA |
| | | | 34 | | AAATCATA (SEQ ID NO: 14) |
| | femC | 111120 | seq | 100 | TTCATCTGTTGTTAAATGCTCTTGTGAGTCATAAA |
| | | | 15 | | TGATTTGTTTAATCC (SEQ ID NO: 15) |
| 10³ | speH | 12653 | seq_ | 100 | AATATTTAGTTAACTCATTAACGTTAGTTTCGTTTT |
| | | | 17 | | GATTTATATAATAA (SEQ ID NO: 16) |
| | rot | 17078 | seq_ | 100 | TTAGAAACACTTTTGGGTGACATTAACTCAATTTT |
| | | | 16 | | CAGCGAGATTGAAAG (SEQ ID NO: 17) |
| | Ipl3_1 | 13588 | seq_ | 100 | ATCACTTTTGCCACAACCTGCTATCGCAAATATTA |
| | | | 34 | | AAATCATA (SEQ ID NO: 18) |
| | femC | 26677 | seq_ | 100 | TTCATCTGTTGTTAAATGCTCTTGTGAGTCATAAA |
| | | | 15 | | TGATTTGTTTAATCC (SEQ ID NO: 19) |
| 10² | speH | 8674 | seq | 100 | AATATTTAGTTAACTCATTAACGTTAGTTTCGTTTT |
| | | | 17 | | GATTTATATAATAA (SEQ ID NO: 20) |
| | rot | 6212 | seq | 100 | TTAGAAACACTTTTGGGTGACATTAACTCAATTTT |
| | | | 16 | | CAGCGAGATTGAAAG (SEQ ID NO: 21) |
| | Ipl3_1 | 15824 | seq | 100 | ATCACTTTTGCCACAACCTGCTATCGCAAATATTA |
| | | | 34 | | AAATCATA (SEQ ID NO: 22) |
| | femC | 3106 | seq | 100 | TTCATCTGTTGTTAAATGCTCTTGTGAGTCATAAA |
| | | | 15 | | TGATTTGTTTAATCC (SEQ ID NO: 23) |
| 10⁰ | speH | 37662 | seq | 100 | AATATTTAGTTAACTCATTAACGTTAGTTTCGTTTT |
| | | | 17 | | GATTTATATAATAA (SEQ ID NO: 24) |
| | rot | 28571 | seq | 100 | TTAGAAACACTTTTGGGTGACATTAACTCAATTTT |
| | | | 16 | | CAGCGAGATTGAAAG (SEQ ID NO: 25) |
| | Ipl3_1 | 31169 | seq_ | 100 | ATCACTTTTGCCACAACCTGCTATCGCAAATATTA |
| | | | 34 | | AAATCATA (SEQ ID NO: 26) |

TABLE 5
Sequences Detected for each Gene Target for Pseudomonas aeruginosa

| | | | SEQ | % | |
|---|---|---|---|---|---|
| Dilution | Gene | CPM | Name | identity | Sequence |
| 10⁷ | pslM | 140 | seq_12 | 100 | TCTGGCACCTGCTGCGCTACGGTCGCGCCATGCAC |
| | | | | | CTGGTCAATGGCGTG (SEQ ID NO: 27) |
| | mucA | 29850 | seq_11 | 100 | AAGGGACCGTGGOGGATGGTCGGTCGCCTGGCGG |
| | | | | | TCGCTGCCTCGGTGAC (SEQ ID NO: 28) |
| | iscR | 392 | seq_31 | 100 | CCGGACCTTGCTGAGCATGCAGCGCCAGGTCGAG |
| | | | | | CATGGCGGTTACGGCG (SEQ ID NO: 29) |
| | fecI_5 | 386 | seq_10 | 100 | AGCACCTGGGCAGTTCGGCGGACGCCGCCGACCT |
| | | | | | CGCCCAGGATACTTTC (SEQ ID NO: 30) |
| | fecI_12 | 107 | seq_33 | 100 | CCGAAGACCTCGCCGCCGAGGCCTTCCTGCAGGT |
| | | | | | CTGGATGCTCCCCGAC (SEQ ID NO: 31) |
| | fecI_11 | 155 | seq_32 | 100 | GCCGAGGACATCGCCTCGGAAACCTTCCTCAAGGT |
| | | | | | CCTTGCCCTGCCGGA (SEQ ID NO: 32) |
| | dksA 1 | 255 | seq_9 | 100 | CTACGGCTGGTGCCAGGAAACCGGCGAGCCGATC |
| | | | | | GGCCTGCGCCGCCTGC (SEQ ID NO: 33) |
| 10⁶ | pslM | 92 | seq_12 | 100 | TCTGGCACCTGCTGCGCTACGGTCGCGCCATGCAC |
| | | | | | CTGGTCAATGGCGTG (SEQ ID NO: 34) |
| | mucA | 29904 | seq_11 | 100 | AAGGGACCGTGGOGGATGGTCGGTCGCCTGGGGG |
| | | | | | TCGCTGCCTCGGTGAC (SEQ ID NO: 35) |
| | iscR | 297 | seq_31 | 100 | CCGGACCTTGCTGAGCATGCAGCGCCAGGTCGAG |
| | | | | | CATGGCGGTTACGGCG (SEQ ID NO: 36) |
| | fecI_5 | 372 | seq_10 | 100 | AGCACCTGGGCAGTTCGGCGGACGCCGCCGACCT |
| | | | | | CGCCCAGGATACTTTC (SEQ ID NO: 37) |
| | fecI_12 | 168 | seq_33 | 100 | CCGAAGACCTCGCCGCCGAGGCCTTCCTGCAGGT |
| | | | | | CTGGATGCTCCCCGAC (SEQ ID NO: 38) |
| | fecI_11 | 225 | seq_32 | 100 | GCCGAGGACATCGCCTCGGAAACCTTCCTCAAGGT |
| | | | | | CCTTGCCCTGCCGGA (SEQ ID NO: 39) |
| | dksA 1 | 357 | seq_9 | 100 | CTACGGCTGGTGCCAGGAAACCGGCGAGCCGATC |
| | | | | | GGCCTGCGCCGCCTGC (SEQ ID NO: 40) |
| 10⁵ | psIM | 299 | seq_12 | 100 | TCTGGCACCTGCTGCGCTACGGTCGCGCCATGCAC |
| | | | | | CTGGTCAATGGCGTG (SEQ ID NO: 41) |
| | mucA | 21357 | seq_11 | 100 | AAGGGACCGTGGOGGATGGTCGGTCGCCTGGCGG |
| | | | | | TCGCTGCCTCGGTGAC (SEQ ID NO: 42) |
| | iscR | 240 | seq_31 | 100 | CCGGACCTTGCTGAGCATGCAGCGCCAGGTCGAG |
| | | | | | CATGGCGGTTACGGCG (SEQ ID NO: 43) |
| 10⁴ | pslM | 1297 | seq_12 | 100 | TCTGGCACCTGCTGCGCTACGGTCGCGCCATGCAC |
| | | | | | CTGGTCAATGGCGTG (SEQ ID NO: 44) |
| | mucA | 18358 | seq_11 | 100 | AAGGGACCGTGGOGGATGGTCGGTCGCCTGGCGG |
| | | | | | TCGCTGCCTCGGTGAC (SEQ ID NO: 45) |
| | fecI_12 | 1054 | seq_30 | 100 | CCGAAGACCTCGCCGCCGAGGCCTTCCTACAGGTC |
| | | | | | TGGATGCTCCCCGAC (SEQ ID NO: 46) |
| 10³ | mucA | 8477 | seq_11 | 100 | AAGGGACCGTGGCGGATGGTCGGTCGCCTGGCGG |
| | | | | | TCGCTGCCTCGGTGAC (SEQ ID NO: 47) |
| | fecI_12 | 1932 | seq_30 | 99.5 | CCGAAGACCTCGCCGCCGAGGCCTTCCTACAGGTC |
| | | | | | TGGATGCTCCCCGAC (SEQ ID NO: 48) |
| | dksA_1 | 1434 | seq_29 | 100 | AGAAGCTGCTCGACAAGATCGACGAAGCGCTGGA |
| | | | | | GCGCCTGGCCCGCGGC (SEQ ID NO: 49) |
| 10² | ptlF_4 | 1348 | seq_28 | 100 | CGCCGGCTGGTGGTGATGACTAGGTTGGTCTTGAG |
| | | | | | GTCCGATOGGATCGG (SEQ ID NO: 50) |
| | fecI 5 | 2110 | seq_10 | 100 | AGCACCTGGGCAGTTCGGCGGACGCCGCCGACCT |
| | | | | | CGCCCAGGATACTTTC (SEQ ID NO: 51) |
| | fecI_11 | 1993 | seq_13 | 100 | GCCGAGGACATCGCCTCGGAAACCTTCCTCAAGGT |
| | | | | | CCTCGCCCTGCCGGA (SEQ ID NO: 52) |

Thereafter, Quantitative Polymerase Chain Reaction Quantitative PCR (qPCR) was performed on the mixture of human sputum, P. aeruginosa and S. aureus at all 8 dilution points. A No Template Control (NTC) was also included. cDNA synthesis was set up and performed for each sample based on the specific requirements of each qPCR assays needs.

The Pseudomonas-specific RT reactions were set up using 8 μl of each dilution point and 2 μl of SuperScript IV Vilo Master Mix. For the No RT reaction, Ing (8 μl) of sample was used as a template with 2 μl SuperScript IV Vilo No RT Ctrl. The samples were run on the thermocycler at 25° C. for 10 min, 50° C. for 10 min, 85° C. for 5 min.

The S. aureus-specific RT reactions were set up using 9.6 μl of each dilution point and 2.4 μl of SuperScript IV VILO Master Mix based on the manufacturer protocol. For the No RT reaction, Ing (9.6 μl) of sample was used a template with 2.4 μl SuperScript IV Vilo No RT Ctrl. The samples were run on the thermocycler at 25° C. for 10 min, 50° C. for 10 min, 85° C. for 5 min and held at 10° C. for less than an hour.

The cDNA samples were taken to qPCR. For P. aeruginosa, TaqMan Fast Advanced Master Mix containing ROX (Catalog #4444556) and a Pseudomonas TaqMan assay (20×) were used. Briefly, for every dilution point, 10 μl of the TaqMan Fast Advanced Master Mix (2×) was mixed with 1 μl of the Pseudomonas TaqMan Assay (20×), 7 μl of nuclease free water and 2 μl of cDNA. Each dilution point was run in triplicate. The plate was run on the QuantStudio 7 following Pub. No. 4444605, Rev. D.

Briefly, PCR was set up as follows: Step (UNG incubation); temperature: 50° C.; time: 2 mins. Step (polymerase activation); temperature 95° C.; time: 2 mins. Step (denature); temperature 95° C.; time: 1 second. Step (anneal/extend); temperature 60° C.; time: 20 seconds. The latter two steps (i.e., denature and anneal/extend) were repeated for 40 cycles.

For S. aureus the TaqMan Staphylococcus aureus Detection Kit (Catalog #4368606) was used. For every dilution point, 3 μl of 10× S. aureus Target Assay Mix was mixed with 15 μl 2× Environmental Master Mix and 12 μl of cDNA. Each dilution point was run in triplicate.

The plate was run on the QuantStudio 7 following Pub. No. 4370454, Rev. C. (protocol accessible at: https://assets.thermofisher.cn/TFS-Assets/LSG/manuals/4370454.pdf, the contents of which are incorporated herein by reference).

Briefly, PCR was set up as follows: Step 1; temperature 95° C.; time: 10 mins. Step 2; temperature 95° C.; time: 15 seconds. Step 3; temperature 60° C.; time: 1 minute. The latter two steps (i.e., Steps 2 and 3) were repeated for 40 cycles.

Detection for qPCR was based on a mean cycle threshold (Ct)<35.

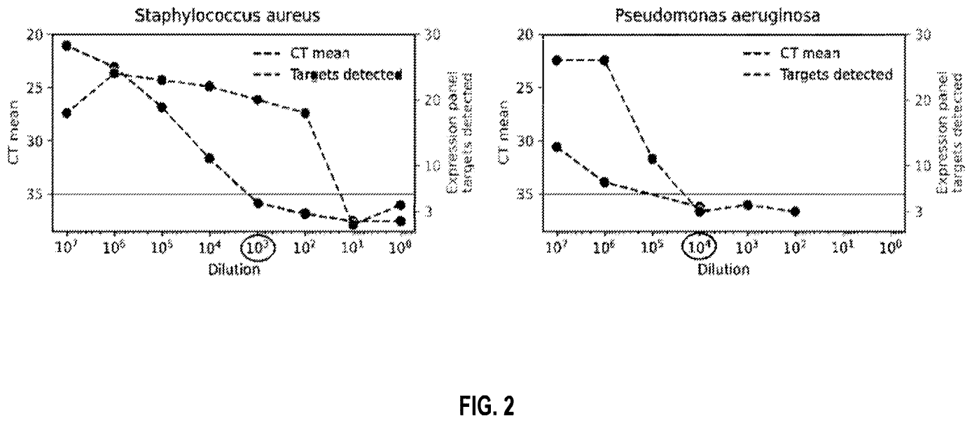

With respect to FIG. 2, the two species in a mixture with human interference (complex sample): S. aureus (left panel) and P. aeruginosa (right panel) in the culture diluted in human sputum. For each panel, the horizontal axis represents the dilution point, with the highest pathogen count on the left and the lowest on the right. For each panel, the left side vertical axis is the cycle threshold (CT) mean of the three replicates for that dilution point from the qPCR assay; the lower the CT value, the more nucleic acid is present. The solid horizontal blue line (at y-axis value: ˜35) denotes a liberal threshold for determining detection of the pathogen based on the CT value (see, for e.g.: Front Microbiol. 2022; 13:820431); when the CT value falls below that line, the qPCR assay would result in non-detection of the pathogen. For each panel, the right side vertical axis is the number of targets detected by the NGS panel. The solid horizontal orange line (at y-axis value ˜3) denotes 3 targets detected, the requirement for detection by the NGS assay; when the number of targets detected falls below that line, the NGS panel would result in non-detection of the pathogen. The LoDs for each method (culture, qPCR, NGS panel) as described above and displayed in FIG. 2 show that the NGS panel had at least an order of magnitude lower LoD (and therefore higher sensitivity) than either culture or qPCR while maintaining perfect specificity (no false positive findings for other pathogens).

Overall, the panel demonstrated a much lower LoD than culture or qPCR while maintaining specificity.

### Example 2. Detection of Fungal Nucleic Acid

Nucleic acid for three fungal species was purchased from the American Type Culture Collection (ATCC): Candida albicans (ATCC-10231D-5), Aspergillus fumigatus (ATCC-MYA-4609D-2), and Candida glabrata (ATCC-2001D-5). The samples were resuspended from lyophilized pellets to a concentration of 20 ng/μl according to their Certificate of Analysis from ATCC. The samples were diluted to ˜4 ng/μl and pooled at equal volumes of 50 μl.

Sequencing library preparation was conducted as described in Example 1 starting from 20 ng DNA rather than RNA. Similarly, Pathogen Gene Detection Analysis was conducted as described in Example 1.

All three fungal species, and no other microbes, were detected in the fungal species pool. Table 6 shows genes for each of Candida albicans (4 genes), Candida glabrata (3 genes), and Aspergillus fumigatus (3 genes), respectively, that were detected by application of the pathogen detection algorithm to the sequencing panel data. Table 7 (Candida albicans), Table 8 (Aspergillus fumigatus), and Table 9 (Candida glabrata) show the detected sequence from the sequencing data and the CPM for the detected sequence for each gene for each species, respectively. Collectively, these tables demonstrate that multiple targets with extremely high sequence similarity to the species called by the pathogen detection algorithm were detected for each of the three species that were present in the single sample (fungal pool) that contained all three of the fungal species. We did not detect any other species in the pool such that the approach yielded 100% sensitivity and 100% specificity for the three fungal species.

TABLE 6
Genes Detected for each Fungal Species in the Fungal Pool

| | |
|---|---|
| Species | Candida albicans |

| | | | | |
|---|---|---|---|---|
| Genes | MEY_{—} | MEY_{—} | MEY_{—} | MEY_{—} |
| | 03361 | 04223 | 05088 | 06242 |

| Species | Aspergillus fumigatus | | |
|---|---|---|---|
| Genes | AFUA_1G05430 | AFUA_8G00910 | AFUA_8G02500 |
| Species | Candida glabrata | | |
| Genes | CAGLOA00187g | CAGLOA00297g | CAGLOA00803g |

*With reference to Table 6, “DET” means detected; and “ND” means not detected.

Table 7 shows the target sequence detected, the percent sequence identity of that sequence to the reference pathogen sequence in the nt database (% identity), and the counts per million (CPM) of that sequence in the NGS sequencing data by the pathogen detection algorithm for each Candida albicans gene in Table 6.

TABLE 7
Candida albicans Sequences Detected for each Gene Target in the Fungal Pool

| | | | SEQ | % | |
|---|---|---|---|---|---|
| Sample | Gene | CPM | Name | Identity | Sequence |
| Fungal | MEY_ | 19094 | seq_ | 100 | GCATTAGCATATAAAACTTTACATCGTAATCAAAAACGAC |
| Pool | 03361 | | 21 | | CACCATTGCC (SEQ ID NO: 53) |
| | MEY_ | 24288 | seq_ | 100 | AGATGCTAAGCCTGGTAATAAAAAGGACTGATTAGTGTC |
| | 04223 | | 22 | | AGTGATATGTG (SEQ ID NO: 54) |
| | MEY_ | 15845 | seq_ | 100 | GAGTTGGACGATGACAACATTTTCTTATGGAATGTTGGTG |
| | 05088 | | 24 | | TAATGGTCTT (SEQ ID NO: 55) |
| | MEY_ | 17000 | seq_ | 99.4 | TATGTTAATCCTAAGACTATGGCATTTGGACAAATTTATC |
| | 06242 | | 35 | | ATGGTGTTCA (SEQ ID NO: 56) |

Table 8 shows the target sequence detected, the percent sequence identity of that sequence to the reference pathogen sequence in the nt database (% identity) and the counts per million (CPM) of that sequence in the NGS sequencing data by the pathogen detection algorithm for each Aspergillus fumigatus gene in Table 6.

TABLE 8
Aspergillus fumigatus Gene Target Sequences Detected in the Fungal Pool for each

| | | | SEQ | % | |
|---|---|---|---|---|---|
| Sample | Gene | CPM | Name | Identity | Sequence |
| Fungal | AFUA_ | 2215 | seq_ | 100 | CTGCAATCCAAGTCCTCCACAACCTTCAACATCAGCAT |
| Pool | 1G05430 | | 33 | | CTTTGGACCTCT (SEQ ID NO: 57) |
| | AFUA_ | 2956 | seq_ | 100 | GGCATAGCATGATGGTAGTCGACCTCATACAGCGTCG |
| | 8G00910 | | 34 | | CCGCGTTCGGGTT (SEQ ID NO: 58) |
| | AFUA_ | 5840 | seq_ | 100 | CCCTGGATACTGGCTCCGAAGGGATGGCTGTCTTGAC |
| | 8G02500 | | 35 | | AACGCCGCGATGA (SEQ ID NO: 59) |

Table 9 shows the target sequence detected, the percent sequence identity of that sequence to the reference pathogen sequence in the nt database (% identity) and the counts per million (CPM) of that sequence in the NGS sequencing data by the pathogen detection algorithm for each Candida glabrata gene in Table 6.

TABLE 9
Candida glabrata Gene Target Sequences Detected in the Fungal Pool for each Gene

| | | | SEQ | % | |
|---|---|---|---|---|---|
| Sample | Gene | CPM | Name | Identity | Sequence |
| Fungal | CAGLOA00187g | 9986 | seq_ | 99.5 | GGCGAGGTAAACAACAAGGATTTACCAGCAATGGTA |
| Pool | | | 36 | | TTAATAAAGATCAA (SEQ ID NO: 60) |
| | CAGLOA00297g | 14417 | seq_ | 100 | ATACTCCTTGTTATCGTAGCATTCCTTTAATTCTGCTT |
| | | | 37 | | CTATTTTTTCTA (SEQ ID NO: 61) |
| | CAGLOA00803g | 14787 | seq_ | 100 | TGGATGATGATGAGCCGATGCAGCGCTATATCCTAC |
| | | | 38 | | AGTCTCTGTTTTCG (SEQ ID NO: 62) |

### Example 3. Comparison to Clinical Microbiology Results in Asthma Clinical Samples

Samples from asthma patients in a clinical research study were used to demonstrate the performance of the panel. To be enrolled and have samples taken, the patients had to be free of any symptoms (e.g., fever) indicative of a respiratory infection. At yearly visits, each patient underwent a bronchoscopy for collection of bronchoalveolar lavage (BAL) fluid in addition to induced sputum collection. The BAL samples were sent to a clinical laboratory for culture and PCR testing. At quarterly clinic visits intervening between the yearly visit that included bronchoscopy, an induced sputum was collected. A total of 97 sputum samples for 17 patients were tested; patients had between 1 and 9 samples each. Of the 17 patients with at least one induced sputum sample available for study, 6 had both bronchoalveolar lavage (BAL) fluid and sputum samples collected at the same clinical visit.

Nucleic acid extraction was performed by the method described for the limit of detection assay in Example 1, above.

To compare clinical microbiology pathogen testing results to the detection results of the NGS panel, the BAL clinical testing results were compared to the panel pathogen detection results in sputum. To obtain the pathogen detection via the panel, the pathogen detection analysis was applied to the NGS reads from the sputum samples. The concordance or discordance between the BAL clinical pathogen detection and the panel detection was recorded.

The comparison of the clinical lab pathogen detection versus the panel detection is shown in Table 10. For each of the 5 positive BAL clinical lab results, the panel detected the pathogen(s) indicated by the clinical lab method (culture or PCR), with the exception of one patient, who had low neutrophils and only oral species detected by the panel (Patient E). Given the sensitivity of the panel to detect S. aureus, demonstrated above, and low BAL % neutrophils, it is concluded that the clinical lab results for Patient E may have been incorrect. In a patient who was negative for pathogen detection in the clinical lab results (Patient C), the panel detected H. parainfluenzae in sputum.

TABLE 10
Comparison of Clinical Pathogen Detection in BAL by culture
or PCR vs. NGS Panel Pathogen Detection in Sputum

| | | | % BAL |
|---|---|---|---|
| Patient | Clinical Lab | | Neutro- |
| Sample | Detection in BAL* | Panel Detection in Sputum | phils |
| A | Rare P. aeruginosa | P. aeruginosa | 24 |
| B | Rare P. aeruginosa | Haemophilus influenzae & | 7 |
| | | P. aeruginosa | |
| C | Negative | Haemophilus | 38 |
| | | parainfluenzae | |
| D | Moderate Haemophilus | Haemophilus influenzae & | 9 |
| | influenzae | P. aeruginosa | |
| E | S. aureus | Only oral species | 1 |
| F | Coronavirus HKU1 | Coronavirus HKU1 | 11 |

*With reference to Table 10, BAL Clinical Lab Detection based on culture for all but Coronavirus HKU1, which was detected by PCR.

Table 11 shows 8 genes for Pseudomonas aeruginosa and whether or not they were detected in each of the patient samples listed in Table 10. Patients A, B and D all had Pseudomonas aeruginosa detected, but with different combinations of gene targets contributing to that detection; Patient A had three of the eight gene targets detected (fec1_5, mucA dksA_1), Patient B had four of the eight gene targets detected (fec1_5, mucA, psIM, dksA_1) and Patient D had six of the eight of the gene targets detected (fec1_1, fec1_5, mucA, psIM, dksA_1, iscR). None of the other patients in Table 10 had any of those eight gene targets detected for Pseudomonas aeruginosa.

TABLE 11
Genes Detected or Not from the NGS Panel for
Pseudomonas aeruginosa in Patients from Table 10

| | Species | | | | | | | |
|---|---|---|---|---|---|---|---|---|
| | Pseudomonas aeruginosa* | | | | | | | |
| | Gene | | | | | | | |
| Patient | fecI_5 | fecI_11 | ptlF_4 | fecI_12 | mucA | pslM | dksA_1 | iscR |
| A | DET | ND | ND | ND | DET | ND | DET | ND |
| B | DET | ND | ND | ND | DET | DET | DET | ND |
| C | ND | ND | ND | ND | ND | ND | ND | ND |
| D | DET | DET | ND | ND | DET | DET | DET | DET |
| E | ND | ND | ND | ND | ND | ND | ND | ND |
| F | ND | ND | ND | ND | ND | ND | ND | ND |

*With reference to Table 11, “DET” means detected; and “ND” means not detected.

As generated by the pathogen detection algorithm, Table 12 shows the target sequence detected, the percent sequence identity of that sequence to the reference pathogen sequence in the nt database (% identity) and the counts per million (CPM) of that sequence in the NGS sequencing data for each Pseudomonas aeruginosa gene target detected in Table 11. Each of the sequences had 100% sequence identity with the Pseudomonas aeruginosa target sequence in the database.

TABLE 12
Pseudomonas aeruginosa Gene Target Sequences Detected in Patients from Table
10.

| | | | SEQ | % | |
|---|---|---|---|---|---|
| Patient | Gene | CFM | Name | identity | Sequence |
| A | dksA_ | 590 | seq_ | 100 | CTACGGCTGGTGCCAGGAAACCGGCGAGCCGATCGGCCT |
| | 1 | | 9 | | GCGCCGCCTGC (SEQ ID NO: 63) |
| | fecl_ | 312 | seq_ | 100 | AGCACCTGGGCAGTTCGGCGGACGCCGCCGACCTCGCCC |
| | 5 | | 10 | | AGGATACTTTC (SEQ ID NO: 64) |
| | mucA | 3654 | seq_ | 100 | AAGGGACCGTGGCGGATGGTCGGTCGCCTGGCGGTCGCT |
| | | | 11 | | GCCTCGGTGAC (SEQ ID NO: 65) |
| B | dksA_ | 2772 | seq_ | 100 | CTACGGCTGGTGCCAGGAAACCGGCGAGCCGATCGGCCT |
| | 1 | | 9 | | GCGCCGCCTGC (SEQ ID NO: 66) |
| | fecl_ | 956 | seq_ | 100 | AGCACCTGGGCAGTTCGGCGGACGCCGCCGACCTCGCCC |
| | 5 | | 10 | | AGGATACTTTC (SEQ ID NO: 67) |
| | mucA | 11889 | seq_ | 100 | AAGGGACCGTGGOGGATGGTCGGTCGCCTGGCGGTCGCT |
| | | | 11 | | GCCTCGGTGAC (SEQ ID NO: 68) |
| | psIM | 67 | seq_ | 100 | TCTGGCACCTGCTGCGCTACGGTCGCGCCATGCACCTGGT |
| | | | 12 | | CAATGGCGTG (SEQ ID NO: 69) |
| D | dksA_ | 19669 | seq_ | 100 | CTACGGCTGGTGCCAGGAAACCGGCGAGCCGATCGGCCT |
| | 1 | | 9 | | GCGCCGCCTGC (SEQ ID NO: 70) |
| | fecl_ | 189 | seq_ | 100 | GCCGAGGACATCGCCTCGGAAACCTTCCTCAAGGTCCTCG |
| | 11 | | 13 | | CCCTGCCGGA (SEQ ID NO: 71) |
| | fecl_ | 7826 | seq_ | 100 | AGCACCTGGGCAGTTCGGCGGACGCCGCCGACCTCGCCC |
| | 5 | | 10 | | AGGATACTTTC (SEQ ID NO: 72) |
| | isCR | 1098 | seq_ | 100 | CCGCCATGCTCGACCTGGCGCTGCATGCTCAGCAAGGTCC |
| | | | 14 | | GGTCTCGCTG (SEQ ID NO: 73) |
| | mucA | 18647 | seq_ | 100 | AAGGGACCGTGGCGGATGGTCGGTCGCCTGGCGGTCGCT |
| | | | 11 | | GCCTCGGTGAC (SEQ ID NO: 74) |
| | psIM | 669 | seq_ | 100 | TCTGGCACCTGCTGCGCTACGGTCGCGCCATGCACCTGGT |
| | | | 12 | | CAATGGCGTG (SEQ ID NO: 75) |

Table 13 shows 3 gene targets for Haemophilus parainfluenzae and whether or not they were detected in each of the patient samples listed in Table 10. Only Patient C had Haemophilus parainfluenzae detected via detection of all three of the 16s gene targets; none of the other samples had any of those three gene targets for Haemophilus parainfluenzae detected.

TABLE 13
Targets Detected or Not from the NGS Panel for Haemophilus
parainfluenzae in Patients from Table 10

| | |
|---|---|
| | Species |
| | Haemophilus parainfluenzae* |
| Patient | Gene |

| Sample | 16s_1 | 16s_2 | 16s_3 |
|---|---|---|---|
| A | ND | ND | ND |
| B | ND | ND | ND |
| C | DET | DET | DET |
| D | ND | ND | ND |
| E | ND | ND | ND |
| F | ND | ND | ND |

*With reference to Table 13, “DET” means detected; and “ND” means not detected.

As generated by the pathogen detection algorithm, Table 14 shows the target sequence detected, the percent sequence identity of that sequence to the reference pathogen sequence in the nt database (% identity) and the counts per million (CPM) of that sequence in the NGS sequencing data for each Haemophilus parainfluenzae gene target detected in Table 13. Two of the sequences had 100% sequence identity with the Haemophilus parainfluenzae target sequence in the database and one had 99.3% sequence identity with the Haemophilus parainfluenzae target sequence in the database.

TABLE 14
Haemophilus parainfluenzae Gene Target Sequences Detected in Patients from
Table 10.

| | | | SEQ | % | |
|---|---|---|---|---|---|
| Patient | Gene | CPM | name | identity | Sequence |
| C | 16s | 22966 | seq_ | 100 | AGCGAGAGTGCGAGCTGGAGCGAATCTCACAAAGTACGTC |
| | | | 54 | | TAAGTCCGGA (SEQ ID NO: 76) |
| | 16s | 21232 | seq_ | 99.3 | AAGCGATAGTGCGAGCTGGAGCGAATCTCACAAAGTACGT |
| | | | 55 | | CTAAGTCCGG (SEQ ID NO: 77) |
| | 16s | 15949 | seq_ | 100 | AAGCGAGAGTGCGAGCTGGAGCGAATCTCACAAAGTACGT |
| | | | 56 | | CTAAGTCCGG (SEQ ID NO: 78) |

Table 15 shows 5 genes for Haemophilus influenzae and whether or not they were detected in each of the patient samples listed in Table 10. Patients B and D both had Haemophilus influenzae detected, but with different combinations of gene targets contributing to that detection; Patient B had three of the five gene targets detected (hgpC, relB, uppB) and Patient D had an additional gene target detected for a total of four of the five gene targets detected (hgpC, relB, uppB, vapD). None of the other patients in Table 10 had any of those five gene targets detected for Haemophilus influenzae.

TABLE 15
Genes Detected or Not from the NGS Panel for
Haemophilus influenzae in Patients from Table 10

| | |
|---|---|
| | Species |
| | Haemophilus influenzae* |
| Patient | Gene |

| Sample | hgpC | relB | uppB | vapD | yafQ_1 |
|---|---|---|---|---|---|
| A | ND | ND | ND | ND | ND |
| B | DET | DET | DET | ND | ND |
| C | ND | ND | ND | ND | ND |
| D | DET | DET | DET | DET | ND |
| E | ND | ND | ND | ND | ND |
| F | ND | ND | ND | ND | ND |

*With reference to Table 15, “DET” means detected; and “ND” means not detected.

As generated by the pathogen detection algorithm, Table 16 shows the target sequence detected, the percent sequence identity of that sequence to the reference pathogen sequence in the nt database (% identity) and the counts per million (CPM) of that sequence in the NGS sequencing data for each Haemophilus influenzae gene target detected in Table 15. Each of the sequences had 100% sequence identity with the Haemophilus influenzae target sequence in the database.

TABLE 16
Haemophilus influenzae Gene Target Sequences Detected in Patients from Table
10.

| | | | SEQ | % | |
|---|---|---|---|---|---|
| Patient | Gene | CPM | Name | identity | Sequence |
| B | hgpC | 410 | seq_1 | 100 | GAGCTTAAGGTTTCAGCTTGACGAAGCCCATCAACCATAAT |
| | | | | | ACCTACACG (SEQ ID NO: 79) |
| | relB | 242 | seq_2 | 100 | CAATATACTGTTTATTAAATCGTTTACCTTTACCCGAAAACT |
| | | | | | ACCAAGAT (SEQ ID NO: 80) |
| | uppB | 97 | seq_3 | 100 | ACTGCCACAGAATGTTATTTATTCGAAGGTGAAGGGCATTT |
| | | | | | GAATAAGTA (SEQ ID NO: 81) |
| D | hgpC | 9071 | seq_4 | 100 | GAGCTTAAGGTTTCAGCTTGACGAAGCCCATCAACCATAAT |
| | | | | | ACCTACGCG (SEQ ID NO: 82) |
| | relB | 4625 | seq_2 | 100 | CAATATACTGTTTATTAAATCGTTTACCTTTACCCGAAAACT |
| | | | | | ACCAAGAT (SEQ ID NO: 83) |
| | uppB | 3209 | seq_3 | 100 | ACTGCCACAGAATGTTATTTATTCGAAGGTGAAGGGCATTT |
| | | | | | GAATAAGTA (SEQ ID NO: 84) |
| | vapD | 9268 | seq_5 | 100 | TTTGTTAGAACACAAGGAAGTTTATATACCAACATGAATGA |
| | | | | | AGATATGGC (SEQ ID NO: 85) |

Table 17 shows 2 gene targets for Coronavirus HKU1 and whether or not they were detected in each of the patient samples listed in Table 10. Only Patient C had Coronavirus HKU1 detected via detection of both of the gene targets; none of the other samples had any of those 2 gene targets for Coronavirus HKU1 detected.

TABLE 17
Genes Detected or Not from the NGS Panel for
Coronavirus HKU1 in Patients from Table 10

Species
Coronavirus HKU1*
Gene

| Patient Sample | Replicase 1B | Spike |
|---|---|---|
| A | ND | ND |
| B | ND | ND |
| C | ND | ND |
| D | ND | ND |
| E | ND | ND |
| F | DET | DET |

*With reference to Table 17, “DET” means detected; and “ND” means not detected.

As generated by the pathogen detection algorithm, Table 18 shows the target sequence detected, the percent sequence identity of that sequence to the reference pathogen sequence in the nt database (% identity) and the counts per million (CPM) of that sequence in the NGS sequencing data for each Coronavirus HKU1 gene target detected in Table 17. One of the sequences had 100% sequence identity with the Coronavirus HKU1 target sequence in the database and the other had 99.5% sequence identity with the Coronavirus HKU1 target sequence in the database.

TABLE 18
Coronavirus HKU1 Gene Target Sequences Detected in Patients from Table 10.

| | | | SEQ | % | |
|---|---|---|---|---|---|
| Patient | Gene | CPM | name | identity | Sequence |
| F | Replicase | 972458 | seq_ | 99.5 | AACACCTGTCATTGAAGCCAAAGCATCTAAAACAAGATCT |
| | 1B | | 25 | | GCTTTTACTT (SEQ ID NO: 86) |
| | Spike | 27542 | seq_ | 100 | TACCCAAACCATAAGAAACATCCACAACATACTCACTTAT |
| | | | 26 | | GCGAGGAACT (SEQ ID NO: 87) |

Collectively, these data show high concordance between Clinical Pathogen Detection and the NGS panel with increased sensitivity and specificity of the NGS panel compared to Clinical Pathogen Detection. All but two of the sequences detected had 100% sequence identity to a reference sequence from the relevant pathogen; the remaining 2 had at least 99.3% sequence identity to a reference sequence.

### Example 4. Pathogen Detection, Immune Response and Antimicrobial Resistance in Asthma Patients

To demonstrate the improved diagnostic function of the NGS panel, as well as the use of the panel to inform therapeutic interventions, the NGS panel was used to detect multiple pathogens (e.g., bacteria, viruses, and fungi), as well as antimicrobial resistance genes, and human immune response genes.

### Anti-Microbial Resistance Gene Detection Data Analysis

To determine whether or not anti-microbial resistance (AMR) genes are expressed in a sample, a very similar algorithm to the pathogen detection analysis was applied. Specifically, starting with the sequencing file that retains only reads that are at least 100 bp long, reads were aligned to the database of expected AMR sequences based on the NGS panel design using Bowtie2 with “very-sensitive” setting, as described and referenced previously herein. Reads that aligned with Bowtie2 were considered potential matches to a target gene sequence in the NGS panel design (target), and those read sequences were then compared to reads that aligned to an AMR gene in the NGS panel design. The total number of reads mapping to a given AMR gene was recorded. An antibiotic class was considered detected in a given sample if at least one gene target from that class was detected (i.e., had at least 20 CPM reads) in that sample. The choice of a subset of the AMR genes in our panel was based on presence of that gene in validated databases such as the National Database for Antibiotic Resistant Organisms (NDARO) with appropriate supporting documentation. We found that six of these genes were ubiquitously expressed in all 97 of the sputum samples, suggesting that they are not appropriate differentiators of AMR activity based on expression levels in sputum. The six genes ubiquitously expressed were cfxA, erm(B), erm(F), erm(X), mef(A) and msr(D). These genes were removed from the AMR detection algorithm despite external evidence for their relevance given these data.

### Host Immune Gene Detection Data Analysis

To determine the read counts for each human immune response gene in the panel, the human immune response CPM counting algorithm started with the filtered sequencing files described herein. A read was retained if it aligned to a human immune target gene sequence (target) that is consistent with the NGS panel design using the same aligner and parameters as used for the Pathogen Detection algorithm and the AMR algorithm, as described herein. The number of reads going to each target was recorded and then standardized by the total number of reads mapping to the human immune portion of the panel to generate the counts per million (CPM) for that gene; this read count table was the input to the immune profile algorithms described below.

The pathogen detection and AMR detection analysis described above was also applied to the NGS reads from each of the sputum samples from 97 asthma patients. The species detected and antibiotic gene classes detected were recorded for each species.

Based on the reference set of 97 sputum samples from the asthma patients, patterns of gene expression that may distinguish samples with and without certain pathogens (or pathogen types) were identified. First, for each gene, the gene expression value for each sample (counts per million) was standardized by subtracting the mean and dividing by the standard deviation of the expression counts across the 97 sputum samples. The median of the standardized expression from the genes contributing to a given pattern was calculated to obtain a score for each sample. To determine which samples were predicted by the algorithm to have a given pathogen or pathogen type, a cut-off value of 1.0 was applied to the scores; samples with a score>1.0 were considered to have presence of an immune response to that pathogen or pathogen type.

To distinguish samples with an immune response to H. influenzae from those without, 10 genes from the immune panel were used (TRAF3, PIK3CD, RUNX1, IFNB1, IFNA5, RORC, IFNG-AS1, IFNA4, IFNA2, RORA); the median of the standardized expression values for the 10 genes are used to calculate a score. Table 18A shows each of the genes, their standardized score, and the H. influenzae score (median of those values). Similarly, 10 genes each were used for a gram negative bacterial immune response score (VAVI, PRKCD, ICOSLG, IRAK2, TICAM1, CSF3, TRAF3, AGER, PTGER4, BCL6). Table 18B shows each of the genes, their standardized score, and the Gram negative bacterial immune response score (median of those values). Similarly, 10 genes were used to define a viral response score (SOCS1, IL13, CSF2, PRKCD, NLRP3, DDX3X, CCR8, TGFB1, CCL17, TNFRSF4). Table 18C shows each of the genes, their standardized score, and the Viral response score (median of those values). Note that for each of these scores, patients can have similar scores based on different combinations individual gene standardized values.

TABLE 18A
Individual Gene Values Contributing to H. influenzae Immune Score

| | | | | | | | | | | | H. |
|---|---|---|---|---|---|---|---|---|---|---|---|
| | | | | | | | | | | | influenzae |
| | | | | | | | IFNG- | | | | Score |
| Patient | TRAF3 | PIK3CD | RUNX1 | IFNB1 | IFNA5 | RORC | AS1 | IFNA4 | IFNA2 | RORA | (Median) |
| 1 | 1.25 | 1.30 | 3.20 | 1.84 | 1.97 | 2.16 | 2.16 | 1.84 | 2.01 | 2.70 | 1.99 |
| 2 | 1.76 | 1.62 | 3.40 | 1.97 | 1.92 | 2.48 | 2.34 | 1.78 | 1.95 | 2.87 | 1.96 |
| 3 | 1.12 | 1.15 | −0.35 | −0.61 | −0.31 | −0.75 | −0.68 | −0.42 | −0.29 | −0.65 | −0.38 |
| 4 | 1.15 | 2.83 | −0.37 | 0.49 | 0.47 | −0.47 | 0.09 | 0.13 | 0.24 | 0.49 | 0.36 |
| 5 | −1.16 | −0.27 | −0.36 | −0.03 | −0.22 | −0.45 | −0.37 | −0.10 | −0.19 | −0.23 | −0.25 |
| 6 | 0.76 | 0.10 | 0.21 | −1.32 | −1.09 | −0.47 | −0.87 | −1.11 | −1.28 | −0.88 | −0.88 |
| 7 | 0.44 | −0.53 | 0.06 | −1.17 | −0.84 | −1.01 | −0.94 | −1.07 | −0.78 | −0.55 | −0.81 |
| 8 | 0.59 | 1.73 | −0.24 | −1.05 | −2.44 | 0.25 | −1.35 | −1.07 | −1.46 | −0.95 | −1.00 |
| 9 | 0.90 | −0.92 | 0.49 | −2.43 | −2.66 | −0.52 | −1.59 | −3.64 | −2.35 | −1.48 | −1.53 |
| 10 | 0.43 | −0.79 | 0.10 | 0.64 | 0.44 | −0.17 | 0.32 | 0.58 | 0.58 | 0.49 | 0.43 |
| 11 | −1.12 | 0.57 | −0.65 | −0.66 | −0.58 | −0.13 | −0.32 | −0.67 | −0.74 | −0.96 | −0.65 |

TABLE 18B
Individual Gene Values Contributing to Gram Negative Bacterial Immune Response Score

| | | | | | | | | | | | Gram |
|---|---|---|---|---|---|---|---|---|---|---|---|
| | | | | | | | | | | | Neg |
| | | | | | | | | | | | Score |
| Patient | VAV1 | PRKCD | ICOSLG | IRAK2 | TICAM1 | CSF3 | TRAF3 | AGER | PTGER4 | BCL6 | (Median) |
| 1 | 2.50 | −1.36 | 1.06 | 1.48 | 0.74 | 3.14 | 1.25 | 1.30 | −0.16 | 2.06 | 1.27 |
| 2 | 2.97 | −0.44 | 1.47 | 1.43 | 0.95 | 1.33 | 1.76 | 1.63 | −0.65 | 0.70 | 1.38 |
| 3 | 0.59 | 3.39 | −0.04 | 1.11 | −0.34 | −0.59 | 1.12 | 0.30 | 1.04 | 1.15 | 0.81 |
| 4 | 1.40 | 1.07 | 2.25 | 3.11 | 2.51 | 3.28 | 1.15 | 2.00 | 2.95 | 2.77 | 2.38 |
| 5 | −0.45 | −1.68 | −0.41 | −0.81 | 0.51 | −0.59 | −1.16 | −0.44 | −0.92 | −0.70 | −0.65 |
| 6 | −0.70 | −0.57 | 0.65 | −0.23 | 0.22 | −0.59 | 0.76 | −0.32 | 0.36 | −1.40 | −0.27 |
| 7 | −0.26 | −0.89 | 0.18 | −0.19 | 0.12 | 0.09 | 0.44 | −0.54 | 0.44 | −1.59 | −0.05 |
| 8 | 0.65 | 0.94 | 1.56 | 0.52 | 0.09 | −0.59 | 0.59 | 1.57 | 1.80 | 1.53 | 0.80 |
| 9 | −1.07 | 2.45 | 0.64 | 1.45 | 0.10 | −0.59 | 0.90 | 1.09 | 1.52 | 1.52 | 1.00 |
| 10 | −0.28 | 0.04 | −0.44 | 1.44 | 0.45 | 1.11 | 0.43 | −0.84 | 0.55 | 0.21 | 0.32 |
| 11 | −0.45 | −0.04 | −1.14 | 0.10 | −1.60 | −0.59 | −1.12 | −0.58 | −0.34 | 1.02 | −0.52 |

TABLE 18C
Individual Gene Values Contributing to Viral Response Immune Score

| | | | | | | | | | | | Virus |
|---|---|---|---|---|---|---|---|---|---|---|---|
| | | | | | | | | | | | Score |
| Patient | SOCS1 | IL13 | CSF2 | PRKCD | NLRP3 | DDX3X | CCR8 | TGFB1 | CCL17 | TNFRSF4 | (Median) |
| 1 | −0.80 | −0.60 | −1.12 | −1.36 | 2.26 | −0.61 | 3.24 | −1.03 | 0.52 | −0.96 | −0.70 |
| 2 | −0.72 | −0.22 | −0.66 | −0.44 | 0.17 | −1.18 | 3.07 | −0.83 | 0.66 | −0.15 | −0.33 |
| 3 | 0.12 | −0.36 | 0.35 | 3.39 | 0.35 | 2.34 | 1.08 | 0.56 | −0.76 | −0.70 | 0.35 |
| 4 | 1.23 | −1.15 | 1.54 | 1.07 | 0.75 | 0.88 | −0.61 | −2.76 | −2.50 | 1.30 | 0.81 |
| 5 | 0.60 | 0.66 | −1.12 | −1.68 | −0.05 | −0.42 | −0.61 | −0.20 | 1.22 | −0.56 | −0.31 |
| 6 | 1.58 | 2.41 | 1.97 | −0.57 | −0.83 | −0.29 | −0.61 | 0.98 | 1.37 | 0.65 | 0.82 |
| 7 | −0.08 | 0.35 | −1.12 | −0.89 | −0.50 | −0.39 | −0.61 | 0.89 | 0.43 | −0.08 | −0.23 |
| 8 | 1.83 | 1.43 | 1.84 | 0.94 | 0.50 | 1.83 | 1.96 | 1.68 | 0.86 | 1.01 | 1.56 |
| 9 | 1.99 | 2.16 | 1.71 | 2.45 | 2.85 | 1.51 | 1.29 | 1.22 | 2.04 | 1.89 | 1.94 |
| 10 | 0.04 | −1.15 | −1.12 | 0.04 | 0.50 | 0.24 | −0.61 | 0.19 | −0.79 | 0.31 | 0.04 |
| 11 | −0.89 | −1.15 | −0.31 | −0.04 | 0.66 | 0.14 | −0.61 | −0.06 | −2.17 | 0.43 | −0.19 |

Table 19 shows a subset of results from two of the three components of the panel (Pathogen Detection. Immune Response) for 11 sputum samples. Bolded cells indicate that the panel found evidence for the pathogen or that type of immune response.

TABLE 19
Paired Pathogen Detection and Immune Response Score for 11 Patients

| | | | | | | | IMMUNE RESPONSE | | |
|---|---|---|---|---|---|---|---|---|---|
| | | | | | | | Gram | | |
| | | | | | | | Negative | | |
| | | | | | | | Bacterial | H. influenzae | Viral |
| | PATHOGEN DETECTION | | | | | | Immune | Immune | Immune |
| Patient | H. influenzae | P. aeruginosa | S. aureus | M. pneumoniae | Virus | Candida albicans | Response | Response | Response |
| Sample | Detected | Detected | Detected | Detected | Detected | Detected | Score | Score | Score |
| 1 | Yes | No | No | No | No | No | 1.27 | 1.99 | −0.70 |
| 2 | Yes | No | No | No | No | No | 1.38 | 1.96 | −0.33 |
| 3 | Yes | No | No | No | No | No | 0.81 | −0.38 | 0.35 |
| 4 | Yes | Yes | No | No | No | No | 2.38 | 0.36 | 0.81 |
| 5 | No | No | Yes | No | No | Yes | −0.65 | −0.25 | −0.31 |
| 6 | No | No | No | Yes | No | No | −0.27 | −0.88 | 0.82 |
| 7 | No | No | No | No | No | Yes | −0.05 | −0.81 | −0.23 |
| 8 | No | No | No | No | Yes | No | 0.80 | −0.99 | 1.56 |
| | | | | | (Corona | | | | |
| | | | | | HKU1) | | | | |
| 9 | No | No | No | No | Yes | No | 0.99 | −1.53 | 1.94 |
| | | | | | (Hep G) | | | | |
| 10 | No | No | No | No | No | No | 0.32 | 0.43 | 0.04 |
| 11 | No | No | No | No | No | No | −0.52 | −0.65 | −0.19 |

Table 20 shows 5 gene targets for Haemophilus influenzae and whether or not they were detected in each of the patient samples listed in Table 19. Four patients had Haemophilus influenzae detected based on at least three of the five gene targets being detected. Patient 1 had three genes detected (hgpC, relB, vapD). Patients 2 and 4 each had the same four of the five genes detected (hgpC, relB, uppB, vapD), while Patient 3 had all five of the five genes detected (hgpC, relB, uppB, vapD, yafQ_1). None of the other samples had any of those five gene targets for Haemophilus influenzae detected.

TABLE 20
Haemophilus influenzae Genes Detected
or Not in Patients in Table 19

| | |
|---|---|
| | Species |
| | Haemophilus influenzae* |
| Patient | Gene |

| Sample | hgpC | relB | uppB | vapD | yafQ_1 |
|---|---|---|---|---|---|
| 1 | DET | DET | ND | DET | ND |
| 2 | DET | DET | DET | DET | ND |
| 3 | DET | DET | DET | DET | DET |
| 4 | DET | DET | DET | DET | ND |
| 5 | ND | ND | ND | ND | ND |
| 6 | ND | ND | ND | ND | ND |
| 7 | ND | ND | ND | ND | ND |
| 8 | ND | ND | ND | ND | ND |
| 9 | ND | ND | ND | ND | ND |
| 10 | ND | ND | ND | ND | ND |
| 11 | ND | ND | ND | ND | ND |

*With reference to Table 20, “DET” means detected; and “ND” means not detected.

As generated by the pathogen detection algorithm, Table 21 shows the target sequence detected, the percent sequence identity of that sequence to the reference pathogen sequence in the nt database (% identity) and the counts per million (CPM) of that sequence in the NGS sequencing data for each Haemophilus influenzae gene target detected. Each of the sequences had 100% sequence identity with the Haemophilus influenzae target sequence in the database.

TABLE 21
Haemophilus influenzae Gene Target Sequences Detected for Patients in Table 19

| | | | SEQ | % | |
|---|---|---|---|---|---|
| Patient | Gene | CPM | Name | identity | Sequence |
| 1 | hgpC | 1670.441 | seq_ | 100 | GAGCTTAAGGTTTCAGCTTGACGAAGCCCATCAACCATA |
| | | | 1 | | ATACCTACACG (SEQ ID NO: 88) |
| | relB | 2059.991 | seq_ | 100 | CAATATACTGTTTATTAAATCGTTTACCTTTACCCGAAAA |
| | | | 2 | | CTACCAAGAT (SEQ ID NO: 89) |
| | vapD | 72284.54 | seq_ | 100 | AAGATTATCATCCAAAGGGCGTTCAAGAGGCTTATACAG |
| | | | 6 | | ACATTC (SEQ ID NO: 90) |
| 2 | hgpC | 2809.969 | seq_ | 100 | GAGCTTAAGGTTTCAGCTTGACGAAGCCCATCAACCATA |
| | | | 4 | | ATACCTACGCG (SEQ ID NO: 91) |
| | relB | 1630.961 | seq_ | 100 | CAATATACTGTTTATTAAATCGTTTACCTTTACCCGAAAA |
| | | | 2 | | CTACCAAGAT (SEQ ID NO: 92) |
| | uppB | 1139.708 | seq_ | 100 | ACTGCCACAGAATGTTATTTATTCGAAGGTGAAGGGCAT |
| | | | 3 | | TTGAATAAGTA (SEQ ID NO: 93) |
| | vapD | 2358.016 | seq_ | 100 | TTTGTTAGAACACAAGGAAGTTTATATACCAACATGAATG |
| | | | 5 | | AAGATATGGC (SEQ ID NO: 94) |
| 3 | hgpC | 103.5307 | seq_ | 100 | GAGCTTAAAGTTTCAGCTTGACGAAGTCCATCAACCATA |
| | | | 7 | | ATACCTACGCG (SEQ ID NO: 95) |
| | relB | 107.0674 | seq_ | 100 | CAATATACTGTTTATTAAATCGTTTACCTTTACCCGAAAA |
| | | | 2 | | CTACCAAGAT (SEQ ID NO: 96) |
| | uppB | 123.4651 | seq_ | 100 | ACTGCCACAGAATGTTATTTATTCGAAGGTGAAGGGCAT |
| | | | 3 | | TTGAATAAGTA (SEQ ID NO: 97) |
| | vapD | 535.9801 | seq_ | 100 | TTTGTTAGAACACAAGGAAGTTTATATACCAACATGAATG |
| | | | 5 | | AAGATATGGC (SEQ ID NO: 98) |
| | yafQ_ | 202.56 | seq_ | 100 | TTGGAAAGGTTGGCGAGATTGCCATATTAAGAATGATCT |
| | 1 | | 8 | | TGTGCTTATTT (SEQ ID NO: 99) |
| 4 | hgpC | 9070.548 | seq_ | 100 | GAGCTTAAGGTTTCAGCTTGACGAAGCCCATCAACCATA |
| | | | 4 | | ATACCTACGCG (SEQ ID NO: 100) |
| | relB | 4625.379 | seq_ | 100 | CAATATACTGTTTATTAAATCGTTTACCTTTACCCGAAAA |
| | | | 2 | | CTACCAAGAT (SEQ ID NO: 101) |
| | uppB | 3209.446 | seq_ | 100 | ACTGCCACAGAATGTTATTTATTCGAAGGTGAAGGGCAT |
| | | | 3 | | TTGAATAAGTA (SEQ ID NO: 102) |
| | vapD | 9267.92 | seq_ | 100 | TTTGTTAGAACACAAGGAAGTTTATATACCAACATGAATG |
| | | | 5 | | AAGATATGGC (SEQ ID NO: 103) |

Table 22 shows 8 gene targets for Pseudomonas aeruginosa and whether or not they were detected in each of the patient samples listed in Table 19. One patient had Pseudomonas aeruginosa detected based on at least three of the 8 gene targets being detected. Patient 4 had six of the eight genes detected (fec1_5, fec1_11, mucA, psIM, dksA_1, iscR). None of the other samples had any of those eight gene targets for Pseudomonas aeruginosa detected.

TABLE 22
Pseudomonas aeruginosa Genes Detected or Not in Patients in Table 19

| | Species | | | | | | | |
|---|---|---|---|---|---|---|---|---|
| | Pseudomonas aeruginosa* | | | | | | | |
| | Gene | | | | | | | |
| Patient | fecI_5 | fecI_11 | ptlF_4 | fecI_12 | mucA | pslM | dksA_1 | iscR |
| 1 | ND | ND | ND | ND | ND | ND | ND | ND |
| 2 | ND | ND | ND | ND | ND | ND | ND | ND |
| 3 | ND | ND | ND | ND | ND | ND | ND | ND |
| 4 | DET | DET | ND | ND | DET | DET | DET | DET |
| 5 | ND | ND | ND | ND | ND | ND | ND | ND |
| 6 | ND | ND | ND | ND | ND | ND | ND | ND |
| 7 | ND | ND | ND | ND | ND | ND | ND | ND |
| 8 | ND | ND | ND | ND | ND | ND | ND | ND |
| 9 | ND | ND | ND | ND | ND | ND | ND | ND |
| 10 | ND | ND | ND | ND | ND | ND | ND | ND |
| 11 | ND | ND | ND | ND | ND | ND | ND | ND |

*With reference to Table 22, “DET” means detected; and “ND” means not detected.

As generated by the pathogen detection algorithm, Table 23 shows the target sequence detected, the percent sequence identity of that sequence to the reference pathogen sequence in the nt database (% identity) and the counts per million (CPM) of that sequence in the NGS sequencing data for each Pseudomonas aeruginosa gene target detected. Each of the sequences had 100% sequence identity with the Pseudomonas aeruginosa target sequence in the database.

TABLE 23
Pseudomonas aeruginosa Gene Target Sequences Detected for Patients in Table
19

| | | | SEQ | % | |
|---|---|---|---|---|---|
| Patient | Gene | CPM | Name | identity | Sequence |
| 4 | dksA_1 | 19668.59 | seq_ | 100 | CTACGGCTGGTGCCAGGAAACCGGCGAGCCGATCGGC |
| | | | 9 | | CTGCGCCGCCTGC (SEQ ID NO: 104) |
| | fecl_11 | 188.791 | seq_ | 100 | GCCGAGGACATCGCCTCGGAAACCTTCCTCAAGGTCCT |
| | | | 13 | | CGCCCTGCCGGA (SEQ ID NO: 105) |
| | fecl_5 | 7826.244 | seq_ | 100 | AGCACCTGGGCAGTTCGGCGGACGCCGCCGACCTCGC |
| | | | 10 | | CCAGGATACTTTC (SEQ ID NO: 106) |
| | iscR | 1098.42 | seq_ | 100 | CCGCCATGCTCGACCTGGCGCTGCATGCTCAGCAAGGT |
| | | | 14 | | CCGGTCTCGCTG (SEQ ID NO: 107) |
| | mucA | 18647.4 | seq_ | 100 | AAGGGACCGTGGOGGATGGTCGGTCGCCTGGCGGTCG |
| | | | 11 | | CTGCCTCGGTGAC (SEQ ID NO: 108) |
| | psIM | 669.3498 | seq_ | 100 | TCTGGCACCTGCTGCGCTACGGTCGCGCCATGCACCTG |
| | | | 12 | | GTCAATGGCGTG (SEQ ID NO: 109) |

Table 24 shows 4 gene targets for Staphylococcus aureus and whether or not they were detected in each of the patient samples listed in Table 19. One patient had Staphylococcus aureus detected based on at least three of the four gene targets being detected. Patient 5 had three of the four genes detected (femC, rot, speH). None of the other samples had any of those four gene targets for Staphylococcus aureus detected.

TABLE 24
Staphylococcus aureus Genes Detected
or Not in Patients in Table 19

| | |
|---|---|
| | Species |
| | Staphylococcus aureus* |
| Patient | Gene |

| Sample | femC | lpl3_1 | rot | speH |
|---|---|---|---|---|
| 1 | ND | ND | ND | ND |
| 2 | ND | ND | ND | ND |
| 3 | ND | ND | ND | ND |
| 4 | ND | ND | ND | ND |
| 5 | DET | ND | DET | DET |
| 6 | ND | ND | ND | ND |
| 7 | ND | ND | ND | ND |
| 8 | ND | ND | ND | ND |
| 9 | ND | ND | ND | ND |
| 10 | ND | ND | ND | ND |
| 11 | ND | ND | ND | ND |

*With reference to Table 24, “DET” means detected; and “ND” means not detected.

As generated by the pathogen detection algorithm, Table 25 shows the target sequence detected, the percent sequence identity of that sequence to the reference pathogen sequence in the nt database (% identity) and the counts per million (CPM) of that sequence in the NGS sequencing data for each Staphylococcus aureus gene target detected. Each of the sequences had 100% sequence identity with the Staphylococcus aureus target sequence in the database.

TABLE 25
Staphylococcus aureus Gene Target Sequences Detected for Patients in Table 19

| | | | SEQ | % | |
|---|---|---|---|---|---|
| Patient | Gene | CPM | Name | identity | Sequence |
| 5 | femC | 44.9 | seq_ | 100 | TTCATCTGTTGTTAAATGCTCTTGTGAGTCATAAATGATTT |
| | | | 15 | | GTTTAATCC (SEQ ID NO: 110) |
| | rot | 247.3 | seq_ | 100 | TTAGAAACACTTTTGGGTGACATTAACTCAATTTTCAGCG |
| | | | 16 | | AGATTGAAAG (SEQ ID NO: 111) |
| | speH | 432.3 | seq_ | 100 | AATATTTAGTTAACTCATTAACGTTAGTTTCGTTTTGATTT |
| | | | 17 | | ATATAATAA (SEQ ID NO: 112) |

Table 26 shows 3 gene targets for Mycoplasma pneumoniae and whether or not they were detected in each of the patient samples listed in Table 19. One patient, Patient 6, had Mycoplasma pneumoniae detected based on all three of the gene targets being detected (galU, hisS, rpoD). None of the other samples had any of those three gene targets for Mycoplasma pneumoniae detected.

TABLE 26
Mycoplasma pneumoniae Genes Detected
or Not in Patients in Table 19

Species
Mycoplasma pneumoniae
Gene

| Patient Sample | galU | hisS | rpoD |
|---|---|---|---|
| 1 | ND | ND | ND |
| 2 | ND | ND | ND |
| 3 | ND | ND | ND |
| 4 | ND | ND | ND |
| 5 | ND | ND | ND |
| 6 | DET | DET | DET |
| 7 | ND | ND | ND |
| 8 | ND | ND | ND |
| 9 | ND | ND | ND |
| 10 | ND | ND | ND |
| 11 | ND | ND | ND |

*With reference to Table 26, “DET” means detected; and “ND” means not detected.

As generated by the pathogen detection algorithm, Table 27 shows the target sequence detected, the percent sequence identity of that sequence to the reference pathogen sequence in the nt database (% identity) and the counts per million (CPM) of that sequence in the NGS sequencing data for each Mycoplasma pneumoniae gene target detected. Each of the sequences had 100% sequence identity with the Mycoplasma pneumoniae target sequence in the database.

TABLE 27
Mycoplasma pneumoniae Gene Target Sequences Detected for Patients in Table 19

| | | | SEQ | % | |
|---|---|---|---|---|---|
| Patient | Gene | CPM | Name | identity | Sequence |
| 6 | galU | 806.614 | seq_ | 100 | TCTTGGTGTTCTTGATCTTTGTGCTTTTGGAGTAAAGCAT |
| | | | 18 | | TTTCCAAGAT (SEQ ID NO: 113) |
| | hisS | 2730.578 | seq_ | 100 | GCGAGCACTACCAGTTTAGTTGTGAAGTGATAGGTGAC |
| | | | 19 | | ACTAATCCAACC (SEQ ID NO: 114) |
| | rpoD | 2765.224 | seq_ | 100 | TTATCACGATTAGAGATGTTTTTGGTGGTTAATTCTTCAA |
| | | | 20 | | TGTCCTCGTC (SEQ ID NO: 115) |

Table 28 shows 2 gene targets for Coronavirus HKU1 and whether or not they were detected in each of the patient samples listed in Table 19. One patient had Coronavirus HKU1 detected based on at least one of the two gene targets being detected. Patient 8 had both genes detected (replicase 1B, spike). None of the other samples had either of those 2 gene targets for Coronavirus HKU1 detected.

TABLE 28
Coronavirus HKU1 Genes Detected or Not in Patients in Table 19

Species
Coronavirus HKU1*
Gene

| Patient Sample | Replicase 1B | Spike |
|---|---|---|
| 1 | ND | ND |
| 2 | ND | ND |
| 3 | ND | ND |
| 4 | ND | ND |
| 5 | ND | ND |
| 6 | ND | ND |
| 7 | ND | ND |
| 8 | DET | DET |
| 9 | ND | ND |
| 10 | ND | ND |
| 11 | ND | ND |

*With reference to Table 28, “DET” means detected; and “ND” means not detected.

As generated by the pathogen detection algorithm, Table 29 shows the target sequence detected, the percent sequence identity of that sequence to the reference pathogen sequence in the nt database (% identity) and the counts per million (CPM) of that sequence in the NGS sequencing data for each Coronavirus HKU1 gene target detected. Each of the sequences had at least 99.5% sequence identity with the Coronavirus HKU1 target sequence in the database.

TABLE 29
Coronavirus HKU1 Gene Target Sequences Detected for Patients in Table 19

| | | | SEQ | % | |
|---|---|---|---|---|---|
| Patient | Gene | CPM | Name | identity | Sequence |
| 8 | Replicase | 918 | seq_ | 99.5 | AACACCTGTCATTGAAGCCAAAGCATCTAAAACAAGATC |
| | 1B | | 25 | | TGCTTTTACTT (SEQ ID NO: 116) |
| | Spike | 26 | seq_ | 100 | TACCCAAACCATAAGAAACATCCACAACATACTCACTTA |
| | | | 26 | | TGCGAGGAACT (SEQ ID NO: 117) |

Table 30 shows the target for Hepatitis G and whether or not it was detected in each of the patient samples listed in Table 19. One patient, Patient 9, had Hepatitis G detected based on the 5′ UTR being detected. None of the other samples had that target for Hepatitis G detected.

TABLE 30
Hepatitis G 5′UTR Detected or Not in Patients in Table 19

| | Species |
|---|---|
| | Hepatitis G |
| | Gene |
| Patient Sample | 5′UTR |
| 1 | ND |
| 2 | ND |
| 3 | ND |
| 4 | ND |
| 5 | ND |
| 6 | ND |
| 7 | ND |
| 8 | ND |
| 9 | DET |
| 10 | ND |
| 11 | ND |

*With reference to Table 30, “DET” means detected; and “ND” means not detected.

As generated by the pathogen detection algorithm, Table 31 shows the target sequence detected, the percent sequence identity of that sequence to the reference pathogen sequence in the nt database (% identity) and the counts per million (CPM) of that sequence in the NGS sequencing data for the Hepatitis G target detected. That sequence had 100% sequence identity with the Hepatitis G target sequence in the database.

TABLE 31
Hepatitis G 5′UTR Gene Target Sequences Detected for Patients in Table 19

| | | | SEQ | % | |
|---|---|---|---|---|---|
| Patient | Gene | CPM | Name | identity | Sequence |
| 9 | 5′UTR | 397 | seq_ | 100 | AGGGTTGGTAGGTCGTAAATCCCGGTCACCTTGGTAGCC |
| | | | 27 | | ACTATAGGTGG (SEQ ID NO: 119) |

Table 32 shows 4 gene targets for Candida albicans and whether or not they were detected in each of the patient samples listed in Table 19. Two patients had Candida albicans detected based on at least three of the four gene targets being detected. Patient 5 had three of the four genes detected (MEY_03361, MEY_04223, MEY_06242). Patient 7 had four of the four genes detected (MEY_03361, MEY_04223, MEY_05088, MEY_06242). None of the other samples had any of those four gene targets for Candida albicans detected.

TABLE 32
Candida albicans Genes Detected or Not in Patients in Table 19

Species
Candida albicans*
Gene

| Patient | MEY_{—} | MEY_{—} | MEY_{—} | MEY_{—} |
|---|---|---|---|---|
| Sample | 03361 | 04223 | 05088 | 06242 |
| 1 | ND | ND | ND | ND |
| 2 | ND | ND | ND | ND |
| 3 | ND | ND | ND | ND |
| 4 | ND | ND | ND | ND |
| 5 | DET | DET | ND | DET |
| 6 | ND | ND | ND | ND |
| 7 | DET | DET | DET | DET |
| 8 | ND | ND | ND | ND |
| 9 | ND | ND | ND | ND |
| 10 | ND | ND | ND | ND |
| 11 | ND | ND | ND | ND |

As generated by the pathogen detection algorithm, Table 33 shows the target sequence detected, the percent sequence identity of that sequence to the reference pathogen sequence in the nt database (% identity) and the counts per million (CPM) of that sequence in the NGS sequencing data for the Candida albicans target detected. Each of the sequences had 100% sequence identity with the Candida albicans target sequence in the database.

TABLE 33
Candida albicans Gene Target Sequences Detected for Patients in Table 19

| | | | SEQ | % | |
|---|---|---|---|---|---|
| Patient | Gene | CPM | Name | identity | Sequence |
| 5 | MEY_ | 434 | seq_21 | 100 | GCATTAGCATATAAAACTTTACATCGTAATCAAAAACGAC |
| | 03361 | | | | CACCATTGCC (SEQ ID NO: 120) |
| | MEY_ | 24 | seq_22 | 100 | AGATGCTAAGCCTGGTAATAAAAAGGACTGATTAGTGTC |
| | 04223 | | | | AGTGATATGTG (SEQ ID NO: 121) |
| | MEY_ | 30 | seq_23 | 100 | TATGTTAATCCTAAGACTATGGCATTTGGCCAAATTTATC |
| | 06242 | | | | ATGGTGTTCA (SEQ ID NO: 122) |
| 7 | MEY_ | 79 | seq_21 | 100 | GCATTAGCATATAAAACTTTACATCGTAATCAAAAACGAC |
| | 03361 | | | | CACCATTGCC (SEQ ID NO: 123) |
| | MEY_ | 127 | seq_22 | 100 | AGATGCTAAGCCTGGTAATAAAAAGGACTGATTAGTGTC |
| | 04223 | | | | AGTGATATGTG (SEQ ID NO: 124) |
| | MEY_ | 81 | seq_24 | 100 | GAGTTGGACGATGACAACATTTTCTTATGGAATGTTGGT |
| | 05088 | | | | GTAATGGTCTT (SEQ ID NO: 125) |
| | MEY_ | 132 | seq_23 | 100 | TATGTTAATCCTAAGACTATGGCATTTGGCCAAATTTATC |
| | 06242 | | | | ATGGTGTTCA (SEQ ID NO: 126) |

For the 7 patients with a bacterial species detected, Table 34 shows the results from the antimicrobial resistance component of the panel. Bolded cells indicate that genes associated with resistance to a particular antibiotic class were detected in that sample. Five of the 7 patients had evidence of AMR for at least one class of antimicrobial treatment. Table 35 shows a gene used to detect AMR to a given class for each patient in Table 34 and whether or not it was detected in each patient. Table 36 shows the specific sequence for each of the gene targets detected for each of the patients with at least one gene AMR target detected.

TABLE 34
Detection or Not of Antimicrobial Resistance for Patients
in Table 19 with a Bacterial Pathogen Found

| | ANTIMICROBIAL RESISTANCE | | | | |
|---|---|---|---|---|---|
| Patient | | BETA- | | | |
| sample | AMINOGLYCOSIDE | LACTAM | FLUOROQUINOLONE | MACROLIDE | SULFONAMIDE |
| 1 | No | No | No | No | No |
| 2 | No | No | No | No | No |
| 3 | No | No | No | No | No |
| 4 | Yes | Yes | No | No | No |
| 5 | No | No | Yes | Yes | Yes |
| 6 | Yes | No | No | No | Yes |
| 7 | Yes | No | No | No | Yes |
| 8 | Yes | No | No | No | No |
| 9 | No | No | No | No | No |
| 10 | No | No | No | No | No |
| 11 | No | No | No | No | No |

TABLE 35
Antimicrobial Resistance Genes Details

| | ANTIMICROBIAL RESISTANCE | | | | | |
|---|---|---|---|---|---|---|
| | AMR class | | | | | |
| | | | | BETA- | | |
| | AMINOGLYCOSIDE | | MACROLIDE | LACTAM | FLUOROQUINOLONE | SULFONAMIDE |
| | Gene | | | | | |
| Patient | aph(3″)- | aac(6′)- | | | | |
| sample | lb | lak | erm(G) | blaPDC | norA | sul2 |
| 1 | ND | ND | ND | ND | ND | ND |
| 2 | ND | ND | ND | ND | ND | ND |
| 3 | ND | ND | ND | ND | ND | ND |
| 4 | ND | DET | ND | DET | ND | ND |
| 5 | ND | ND | DET | ND | DET | DET |
| 6 | DET | ND | ND | ND | ND | DET |
| 7 | DET | ND | ND | ND | ND | DET |
| 8 | DET | ND | ND | ND | ND | ND |
| 9 | ND | ND | ND | ND | ND | ND |
| 10 | ND | ND | ND | ND | ND | ND |
| 11 | ND | ND | ND | ND | ND | ND |

*With reference to Table 35, “DET” means detected; and “ND” means not detected.

TABLE 36
Sequences Detected for each AMR Gene Target for each Patient in Table 19 with
a Bacterial Pathogen and at least one AMR Gene Detected

| | | | | SEQ | |
|---|---|---|---|---|---|
| Patient | Class | Gene | CPM | Name | Sequence |
| 4 | AMINOGLYCOSIDE | aac(6′)- | 38 | seq_ | CTTCCACGCGACTGTCCGAAGCCAGTTCG |
| | | lak | | 53 | CGGCAGCCCGCGTCGCGCGTC (SEQ ID NO: 127) |
| | BETA-LACTAM | blaPDC | 121 | seq_ | CGCCTACGTGGCGTTCGTCCCGGGCCGCGACCTGGGACT |
| | | | | 50 | GGTGA (SEQ ID NO: 128) |
| 5 | FLUOROQUINOLONE | norA | 444 | seq_ | TTTTTGCTAATGGCTATTGGTCAATAATGTT |
| | | | | 45 | AATCAGTTTTGTTGTCTTC (SEQ ID NO: 129) |
| | MACROLIDE | erm(G) | 49 | seq_ | GAAACTTTTGTAATGAAATGGGTTAACAAA |
| | | | | 46 | GAGTATGAAAAACTGTTTAC (SEQ ID NO: 130) |
| | SULFONAMIDE | sul2 | 66 | seq_ | GAGTTTGGCAGATGATTTCGCCAATTGCGGATAGAACGCA |
| | | | | 44 | GCGTCTGGAA (SEQ ID NO: 131) |
| 6 | AMINOGLYCOSIDE | aph(3″)- | 400 | seq_ | CCGGTAAGAAGTCGGGATTGACGGCATTGCGGGACACCA |
| | | lb | | 42 | CATCAACGGCG (SEQ ID NO: 132) |
| | SULFONAMIDE | sul2 | 390 | seq_ | CGCCTGTTTCGTCCGACACAGAAATCGCGCGTATCGCGCC |
| | | | | 43 | GGTGCTGGAC (SEQ ID NO: 133) |
| 7 | AMINOGLYCOSIDE | aph(3″)- | 32 | seq_ | CCGGTAAGAAGTCGGGATTGACGGCATTGCGGGACACCA |
| | | lb | | 42 | CATCAACGGCG (SEQ ID NO: 134) |
| | SULFONAMIDE | sul2 | 78 | seq_ | GAGTTTGGCAGATGATTTCGCCAATTGCGGATAGAACGCA |
| | | | | 44 | GCGTCTGGAA (SEQ ID NO: 135) |
| 8 | AMINOGLYCOSIDE | aph(3″)- | 23 | seq_ | GCCTCATTTGGCTCAAAGGTCGAGGTGTGGCTTGCCCCGA |
| | | lb | | 47 | GGTGATCAAC (SEQ ID NO: 136) |

The three components of the panel refine both diagnosis and treatment options beyond what any one aspect of the panel could provide. Specifically, the immune response scores distinguish between commensal and pathogenic presence of a microbe or microbes.

For example, detection of H. influenzae and/or P. aeruginosa by next-generation sequencing in an asthma patient could lead to either diagnosis of no infection and therefore no treatment if the panel doesn't indicate that the presence of H. influenzae and/or P. aeruginosa is causing an immune response that is contributing to asthma symptoms, or diagnosis of infection and subsequent treatment with one of several antibiotics to which H. influenzae and/or P. aeruginosa is sensitive if the panel indicates that the infection is likely contributing to an immune response that drives asthma symptoms. Antibiotic treatment may be beneficial for these patients.

Additionally, information on the host immune response can refine the diagnosis of detection of the pathogen. If the immune response is indicative of response to the pathogen, then a diagnosis of infection may be made. If the immune response is not indicative of infection by the pathogen, then a diagnosis of no infection (i.e., commensal presence of the microbial species) may be made. After diagnosis of infection rather than commensal presence of the microbial species, the antimicrobial resistance information can refine the available treatment options for a given patient.

For example, Table 19 demonstrates a correlation between some pathogen detection and immune response in many cases. For example, when a virus was not detected, the viral immune response score for that type of pathogen was also <1 in all cases. Similarly, the H. influenzae immune score was negative for samples that did not have H. influenzae detected, and the gram-negative bacterial immune response scores were <1 when no gram-negative bacterial pathogen was detected in all cases in Table 19.

However, in other cases, a lack of correlation between pathogen detection and immune response informs an appropriate diagnosis. For example, when comparing Patients 1 and 2 to Patients 3 and 4 in Table 19, all four patients had H. influenzae detected in sputum. However, Patients 1 and 2 had a gram negative bacterial immune response score>1.0, indicative of a gram-negative bacterial infection, and a score indicative for an immune response specific to H. influenzae (>1.0) while Patient 3 had neither immune response (scores less <1.0 for both gram-negative bacterial immune and H. influenzae response). Patient 4 had an immune response to a gram negative bacterial pathogen (score>1.0), but not an H. influenzae response (score<1). Therefore, a physician could use this information to make a diagnosis of an infection with H. influenzae in Patients 1 and 2, but not Patients 3 and 4. As shown in Table 19, in addition to H. influenzae, Patient 4 also had P. aeruginosa detected, a gram negative bacterial pathogen. Patient 4 did have an immune response to a gram negative bacterial pathogen (score>1). Therefore, a physician could use this information to make a diagnosis of infection with P. aeruginosa as opposed to H. influenzae for Patient 4. Further, after diagnosing each of Patients 1 and 4 with a gram-negative infection (H. influenzae and P. aeruginosa, respectively), observing antimicrobial resistance detection informs potential treatment options for these two patients. Specifically, as shown in Table 34, Patient 1 did not show expression of resistance genes to any of the antibiotics shown. However, Patient 4 showed resistance to the aminoglycoside and beta-lactam classes, both of which are active against gram negative bacterial pathogens and hence would have been potential treatment options for both patients. Based on this information from the panel, the physician may determine that those two classes are available for Patient 1, but not indicated for Patient 4.

As described above, multiple genes are detected in order to determine detection of a pathogen. Observing expression of multiple genes that could potentially be detected results in greater sensitivity compared to choosing one target gene, as is often standard for qPCR. For instance, for H. influenzae (Table 20), among the patients who tested positive, there were different combinations of genes detected (hgpC, relB, uppB, vapD, yafQ_1). In Patient 1, the panel detected all genes except uppB. If the uppB gene had been selected for a qPCR assay, Patient 1 could be misdiagnosed as not having an H. influenzae infection. But it is clear by the detection of four other genes (hgpC, relB, vapD and yafQ_1) that the pathogen is present. Similarly, a misdiagnosis for C. albicans could have been made for Patient 5 if another assay was used. As shown in Table 32, the panel showed no presence of the MEY_05088 gene in the patient sample. In a qPCR assay targeting the MEY_05088 gene only, the patient could be misdiagnosed as not having a C. albicans infection. However, the panel indicated expression of three other C. albicans genes (MEY_03361, MEY_04223, and MEY_06242), yielding a more accurate diagnosis.

1. A system for detecting infection, the system comprising:
a pathogen detection component for detecting a pathogen;
a host immune response component for detecting a host immune response associated with the pathogen; and
an antimicrobial resistance component for detecting an antimicrobial resistance associated with the pathogen.

2. The system of claim 1, wherein the pathogen detection component detects mRNA for species-specific detection of one or more pathogens selected from the group consisting of bacterial pathogens, viral pathogens, and fungal pathogens.

3. The system of claim 1, wherein the host immune response component detects a human immune gene expression profile.

4. (canceled)

5. A panel for detecting a pathogen and an associated biological response in an ex vivo biological sample, the panel comprising:
a pathogen detection component for amplifying at least a portion of a gene associated with the pathogen; and
an immune-response component for amplifying at least a portion of a gene associated with an immune response associated with the pathogen.

6. The panel of claim 5, further comprising an antimicrobial resistance component for amplifying at least a portion of a gene associated with an antimicrobial resistance to the pathogen.

7. The panel of claim 5, wherein the pathogen is a bacteria, a virus, or a fungus.

8. The panel of claim 7, wherein the bacteria is Staphylococcus aureus, Pseudomonas aeruginosa, Haemophilus parainfluenzae, Haemophilus influenzae, Mycoplasma pneumoniae, or a mutant thereof.

9. The panel of claim 7, wherein the fungus is Candida albicans, Aspergillus fumigatus, Candida glabrata, or a mutant thereof.

10. The panel of claim 7, wherein the virus is a Hepatitis virus or a Coronavirus, or a mutant thereof.

11. The panel of claim 7, wherein the bacteria is Staphylococcus aureus, and the gene is selected from one or more of dksA_1, fec1_11, fec1_12, fec1_5, iscR, mucA, psIM, pTIF-4, speH, rot, femC, and IpI3_1.

12. The panel of claim 7, wherein the bacteria is Pseudomonas aeruginosa, and the gene is selected from one or more of psiM, mucA, iscR, fec1_5, fec1_12, fec1_11, ptIF_4, and dksA_1.

13. The panel of claim 7, wherein the bacteria is Haemophilus parainfluenzae, and the gene is selected from one or more of 16s_1, 16s_2, and 16s_3.

14. The panel of claim 7, wherein the bacteria is Haemophilus influenzae, and the gene is selected from one or more of hgpC, relB, uppB, vapD, and yafQ_1.

15. The panel of claim 7, wherein the bacteria is Mycoplasma pneumoniae, and the gene is selected from one or more of galU, hisS, and rpoD.

16. The panel of claim 7, wherein the fungus is Candida albicans, and the gene is selected from one or more of MEY_03361, MEY_04223, MEY_05088, and MEY_06242.

17. The panel of claim 7, wherein the fungus is Aspergillus fumigatus, and the gene is selected from one or more of AFUA_1G05430, AFUA_8G00910, and AFUA_8G02500.

18. The panel of claim 7, wherein the fungus is Candida glabrata, and the gene is selected from one or more of CAGLOA00187g, CAGLOA00297g, and CAGLOA00803g.

19. The panel of claim 7, wherein the virus is Coronavirus HKU1, and the gene is selected from one or more of Replicase 1B and Spike.

20. The panel of claim 7, wherein the virus is Hepatitis G, and the gene is 5′UTR.

21. The panel of claim 6, wherein the gene associated with an antimicrobial resistance is selected from one or more of aph(3″)=1b, aac(6′)-lak, blaPDC, erm(G), norA, and sul2.

22. The panel of claim 11, wherein the sequence of the gene is at least 80%, or at least 90%, or at least 95% identical to any one of SEQ ID NOs: 1-26, or 110-112.

23. The panel of claim 12, wherein the sequence of the gene is at least 80%, or at least 90%, or at least 95% identical to any one of SEQ ID NOs: 27-52, 63-75, or 104-109.

24. The panel of claim 16, wherein the sequence of the gene is at least 80%, or at least 90%, or at least 95% identical to any one of SEQ ID NOs: 53-56, or 120-126.

25. The panel of claim 17, wherein the sequence of the gene is at least 80%, or at least 90%, or at least 95% identical to any one of SEQ ID NOs: 57-59.

26. The panel of claim 18, wherein the sequence of the gene is at least 80%, or at least 90%, or at least 95% identical to any one of SEQ ID NOs: 60-62.

27. The panel of claim 13, wherein the sequence of the gene is at least 80%, or at least 90%, or at least 95% identical to any one of SEQ ID NOs: 76-78.

28. The panel of claim 15, wherein the sequence of the gene is at least 80%, or at least 90%, or at least 95% identical to any one of SEQ ID NOs: 79-85.

29. The panel of claim 19, wherein the sequence of the gene is at least 80%, or at least 90%, or at least 95% identical to any one of SEQ ID NOs: 86-87, or 116-118.

30. The panel of claim 14, wherein the sequence of the gene is at least 80%, or at least 90%, or at least 95% identical to any one of SEQ ID NOs: 88-103.

31. The panel of claim 15, wherein the sequence of the gene is at least 80%, or at least 90%, or at least 95% identical to any one of SEQ ID NOs: 113-115.

32. The panel of claim 20, wherein the sequence of the gene is at least 80%, or at least 90%, or at least 95% identical to SEQ ID NO: 119.

33. The panel of claim 5, wherein the immune-response component is associated with a bacterial pathogen, a viral pathogen, or a fungal pathogen.

34. The panel of claim 33, wherein the immune-response component is associated with a bacterial pathogen, and the gene is selected from TRAF3, PIK3CD, RUNX1, IFNB1, IFNA5, RORC, IFNG-AS1, IFNA4, IFNA2, and RORA.

35. The panel of claim 33, wherein the immune-response component is associated with a bacterial pathogen, and the gene is selected from VAVI, PRKCD, ICOSLG, IRAK2, TICAM1, CSF3, TRAF3, AGER, PTGER4, and BCL6.

36. The panel of claim 33, wherein the immune-response component is associated with a viral pathogen, and the gene is selected from SOCS1, IL13, CSF2, PRKCD, NLRP3, DDX3X, CCR8, TGFB1, CCL17, and TNFRSF4.

37. The panel of claim 21, wherein the sequence of the gene is at least 80%, or at least 90%, or at least 95% identical to any one of SEQ ID NOs: 127-136.
